(19) United States
(12) Patent Application Publication
(10) Pub. No.: US 2003/0049731 A1
(43) Pub. Date: Mar. 13, 2003

(54) Engineered plasmids and their use for in situ production of genes

(76) Inventors: Katherine S. Bowdish, Del Mar, CA (US); Shana Frederickson, Solana Beach, CA (US); Ying-Chi Lin, San Diego, CA (US); Mark Renshaw, San Diego, CA (US); Martha Wild, San Diego, CA (US); John McWhirter, San Diego, CA (US)

Correspondence Address: Mark Farber, Alexion Pharmaceuticals, Inc., 352 Knotter Drive, Cheshire, CT, 06410, US

(21) Appl. No.: 10/006,591

(22) Filed: Dec. 5, 2001

Related U.S. Application Data
(60) Provisional application No. 60/251,440, filed on Dec. 5, 2000.

(51) Int. Cl. C12P 21/02; C12P 19/34; C12N 5/06

(52) U.S. Cl. 435/69.1; 435/91.2; 435/320.1; 435/326

(57) ABSTRACT

Nucleic acid sequences encoding at least a portion of a polypeptide are directly incorporated into a plasmid by DNA polymerization or reverse transcription of a nucleic acid template. In particularly preferred embodiments, nucleic acid sequences encoding at least a portion of an antibody are directly incorporated into a plasmid by reverse transcription of messenger RNA (mRNA).

### RELATED APPLICATIONS

This application claims priority to U.S. Provisional Application No. 60/251,440 filed Dec. 5, 2000, the disclosure of which is incorporated herein by reference.

### BACKGROUND

1. Technical Field

This disclosure relates to the direct incorporation of genes into a plasmid vector by DNA polymerization or reverse transcription of nucleic acid encoding a polypeptide. In certain embodiments, this disclosure relates to the direct incorporation of antibody genes into a plasmid vector by reverse transcription of messenger RNA (mRNA) encoding at least a part of an antibody.

2. Related Art

IgG antibodies are made up of four polypeptide chains, comprising two identical light chains and two identical heavy chains, and can be thought of as forming a flexible Y-shaped structure. Each of the four chains has a variable (V) region at its amino terminus, which contributes to the antigen-binding site, and a constant (C) region, which in the heavy chain determines the isotype and hence the functional properties of the antibody. The light chains are bonded to the heavy chains by many non-covalent interactions and by disulfide bonds, and the V regions of the heavy and light chains pair to generate two identical antigen-binding sites, which lie at the tips of the arms of the Y. The possession of the two antigen-binding sites allows antibody molecules to crosslink antigens. The trunk of the Y, or Fc fragment, is composed of the two carboxy-terminal domains of the two heavy chains. Joining the arms of the Y to the trunk are the flexible hinge regions. The Fc fragment and the hinge regions differ in antibodies of different isotypes, thus determining their functional properties. However, the overall organization of the domains is similar in all isotypes.

Direct cloning of antibody DNA has been achieved by traditional cDNA methods as well as by the polymerase chain reaction (PCR). PCR involves providing a first primer that hybridizes to the sense strand of the dsDNA encoding an antibody and a second primer that hybridizes to the anti-sense strand of the dsDNA encoding an antibody. At elevated temperatures, the two strands of the DNA separate. Upon cooling, the two primers each attach to one of the two complementary DNA strands and complementary strands are produced for each of the two starting strands. In this manner, two double strands each having an original single strand and a newly formed strand that is complementary thereto are produced. By cycling between the elevated temperature which separates the double strands and the cooler temperature which allows a primer to hybridize to each of the sense and anti-sense strands, the PCR process is repeated until the desired number of copies of the dsDNA are obtained. The preparation of an antibody library and use of PCR to clone antibodies or antibody fragments is disclosed, for example, in EPO 386 684B1, the disclosure of which is incorporated herein by reference.

The dsDNA produced via PCR can be recovered and ligated into a plasmid. To do this, double stranded antibody DNA produced by PCR is isolated, can be cleaved and mixed with a cleaved, double stranded plasmid in a solution containing a ligase. Because the ligation depends on the appropriate ends of the dsDNA and the plasmid achieving physical proximity in the solution, there is an inefficiency inherent in this process resulting in less than all the dsDNA prepared finding its way into the plasmid.

It would be desirable to provide a method of incorporating antibody genes directly into a plasmid. A method which forms an antibody gene in situ within a plasmid would be desirable since it would not require ligation of previously prepared dsDNA into a double stranded plasmid, and thus would not rely on solution kinetics and would thereby improve efficiency and avoid the unnecessary loss of dsDNA.

### SUMMARY

It has now been found that nucleic acid sequences encoding at least a portion of a polypeptide can be directly incorporated into a plasmid by DNA polymerization or reverse transcription of a nucleic acid template. In particularly preferred embodiments, nucleic acid sequences encoding at least a portion of an antibody can be directly incorporated into a plasmid by reverse transcription of messenger RNA (mRNA).

In one aspect, plasmids into which nucleic acid sequences encoding at least a portion of a polypeptide can be incorporated are disclosed herein. These plasmids are engineered to contain two template annealing sequences, namely, a downstream primer sequence capable of annealing to a first portion of a nucleic acid template, such as, for example mRNA encoding at least a portion of an antibody, an upstream collar sequence capable of annealing to a second portion of the template and at least one restriction site located between the two template annealing sequences.

A method of using such engineered plasmids is also described wherein the plasmid is cleaved at the one or more restriction sites, and exposed to a nucleic acid template encoding at least a portion of a polypeptide in an aqueous environment containing enzymes and nucleotides under conditions suitable for DNA polymerization or reverse transcription (depending on whether the template is DNA or mRNA) to take place. In this manner, a nucleic acid strand that is complementary to the template is formed in situ between the first and second primers. Ligation closes the previously cleaved plasmid to produce a single stranded DNA plasmid vector containing a nucleic acid sequence encoding at least a portion of a polypeptide such as, for example, a light chain and/or a heavy chain of an antibody.

The plasmid vector so produced can be transformed into a host cell and amplified.

### BRIEF DESCRIPTION OF THE DRAWINGS

FIG. 1A is a schematic view of an engineered plasmid in accordance with this disclosure into which a nucleic acid sequence can be directly incorporated;

FIG. 1B is a schematic view of an alternative embodiment of an engineered plasmid in accordance with this disclosure into which a nucleic acid sequence can be directly incorporated;

FIG. 2 is a map of the pRL5-CAT vector;

FIGS. 3A-C schematically illustrate the sequence (SEQ. ID. NO: 1) of the single strand pRL5-CAT vector useful as the starting plasmid in accordance with this disclosure;

FIGS. 4A-T schematically illustrate plasmid pRL5-CAT, including the double stranded nucleic acid sequence, (comprising SEQ. ID. NO: 1 and SEQ. ID. NO: 2) and domains corresponding to particular genes.

FIG. 5A schematically illustrates a process for cleaving an engineered plasmid in accordance with this disclosure;

FIG. 5B schematically illustrates the cleaved plasmid of FIG. 5A annealed to a template encoding at least a part of a polypeptide;

FIG. 5C schematically illustrates the result of reverse transcription and ligation to produce a plasmid vector having a nucleic acid sequence encoding at least a part of a polypeptide that has been formed in situ within the cleaved plasmid; and

FIGS. 6A and B schematically illustrate engineered plasmids in accordance with Example 1 herein.

FIG. 7 schematically illustrates an alternative process for forming a nucleic acid sequence directly into a plasmid.

### DETAILED DESCRIPTION OF PREFERRED EMBODIMENTS

While the following description is presented with specific reference to mRNA encoding at least a portion of an antibody, it should be understood that the inventions described herein are not so limited. Thus, for example, mRNA encoding a polypeptide other than an antibody could be used as the template for reverse transcription. As another example, cDNA could be used instead of mRNA as the template and DNA polymerization used to incorporate a complementary nucleic acid directly into the engineered plasmid. Those skilled in the art will be capable of readily making the necessary adjustments to the materials and methods described herein to carry out such variations without undue experimentation.

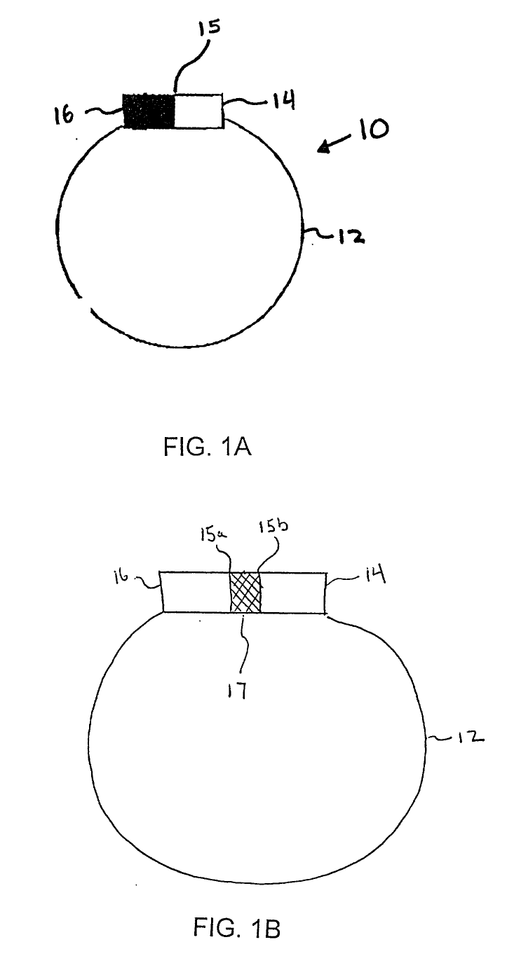

A nucleic acid sequence encoding at least a portion of an antibody can be directly incorporated into an engineered plasmid in accordance with the methods described herein. As shown schematically in FIG. 1A, the engineered plasmid 10 into which the nucleic acid sequence is incorporated is preferably a single strand, circular nucleic acid 12 engineered to contain two template annealing sequences, namely, a downstream primer sequence 14, and an upstream collar sequence 16 separated by at least one restriction site 15.

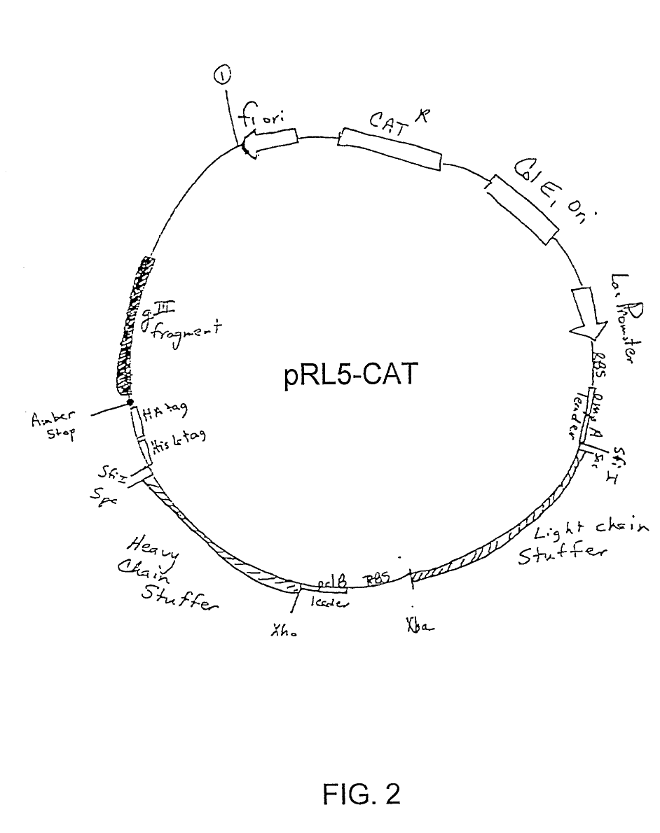

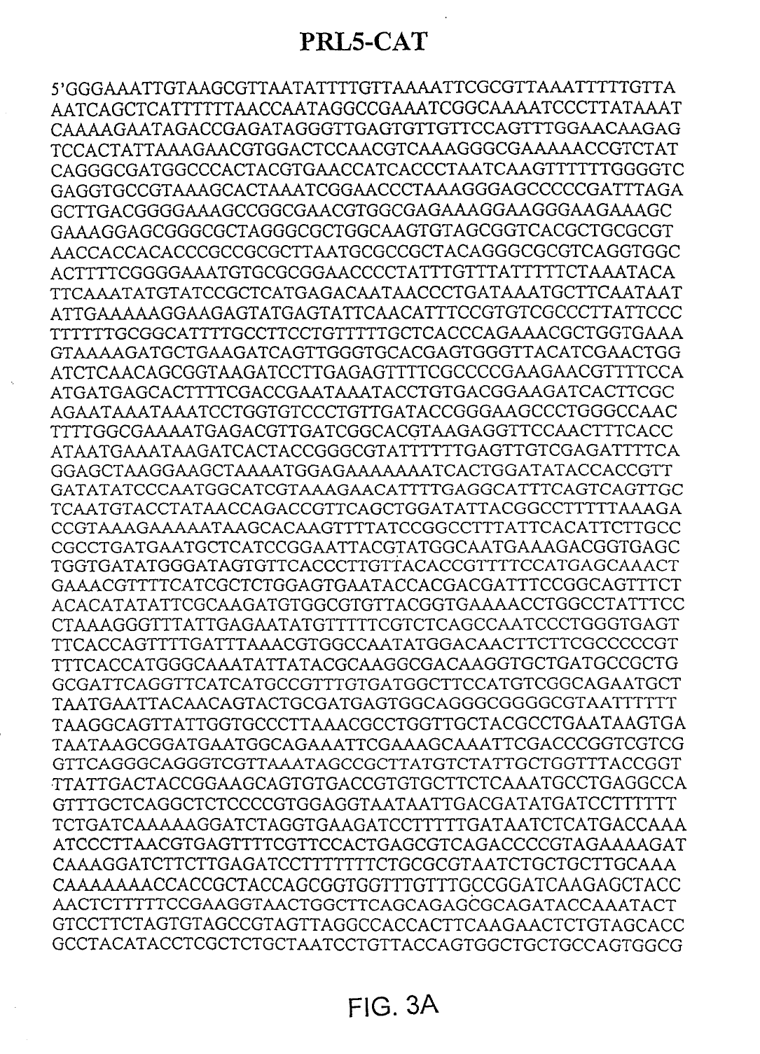

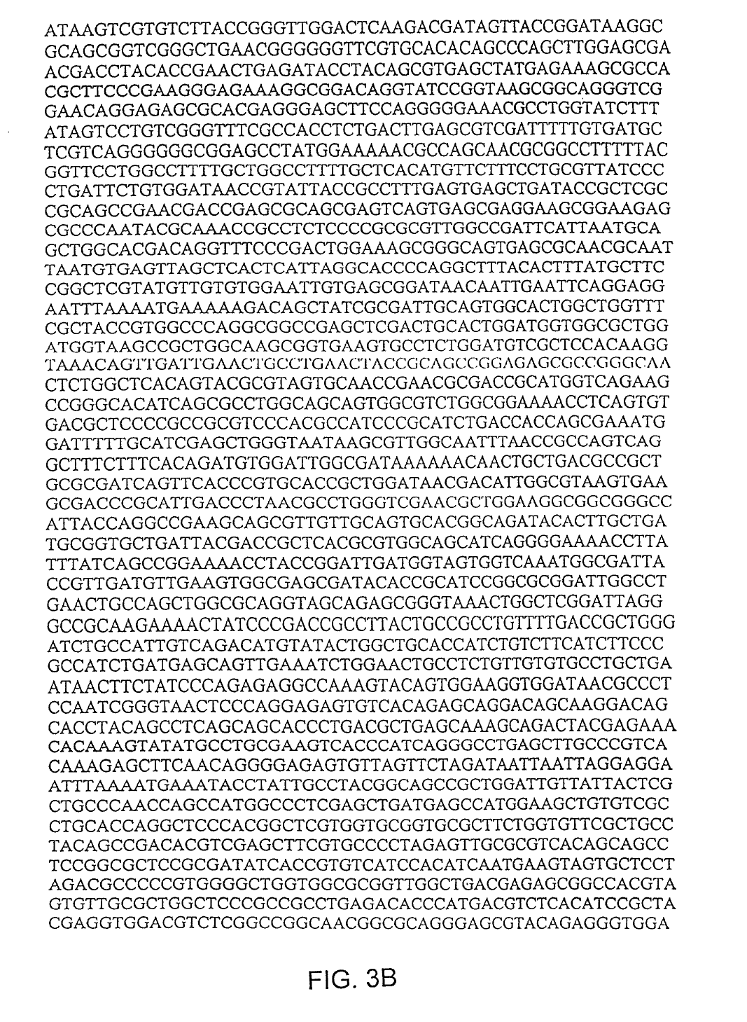

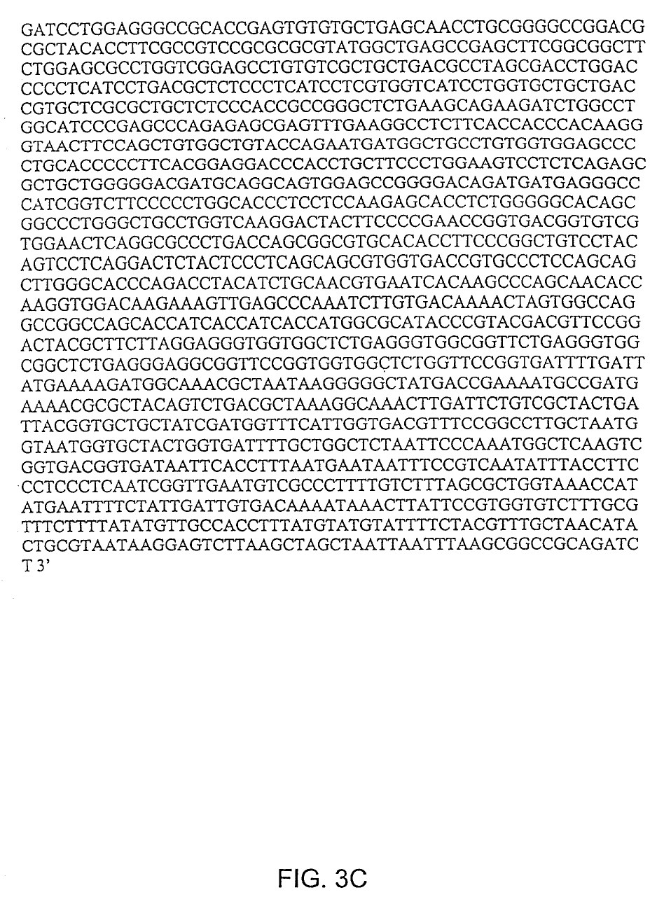

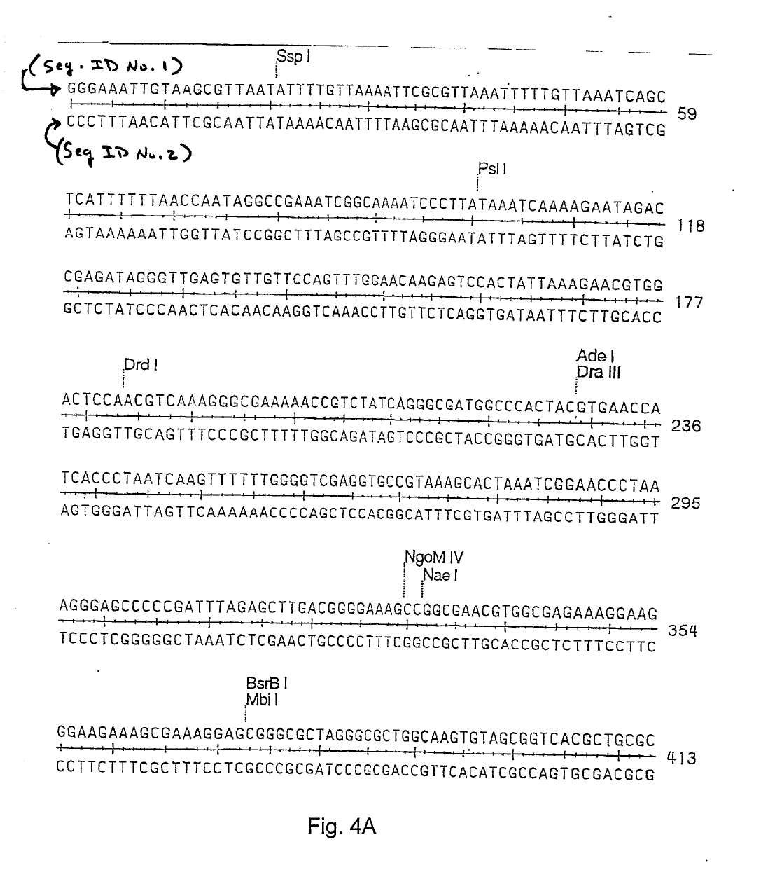

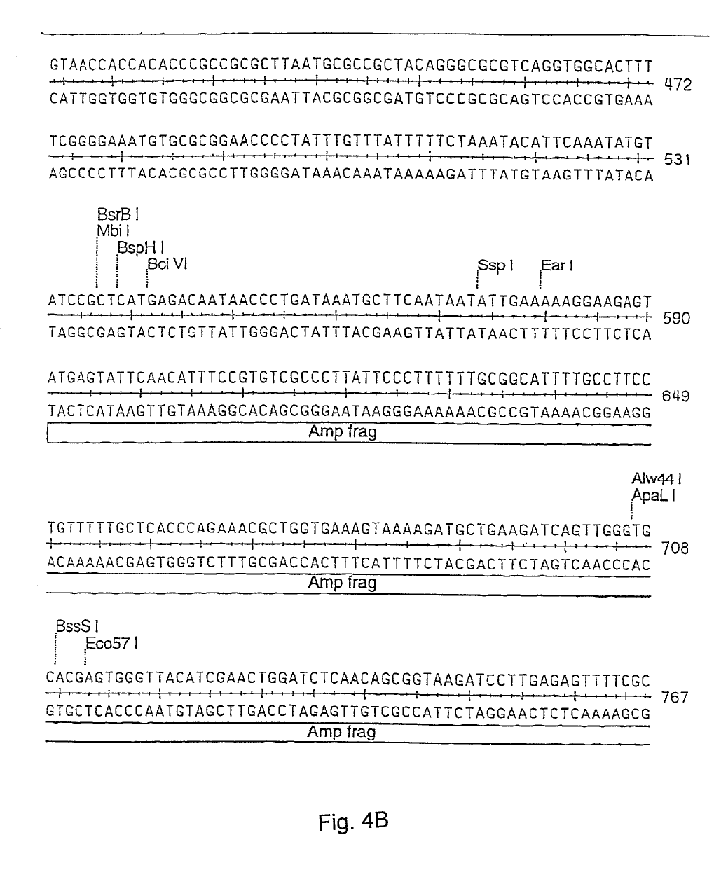

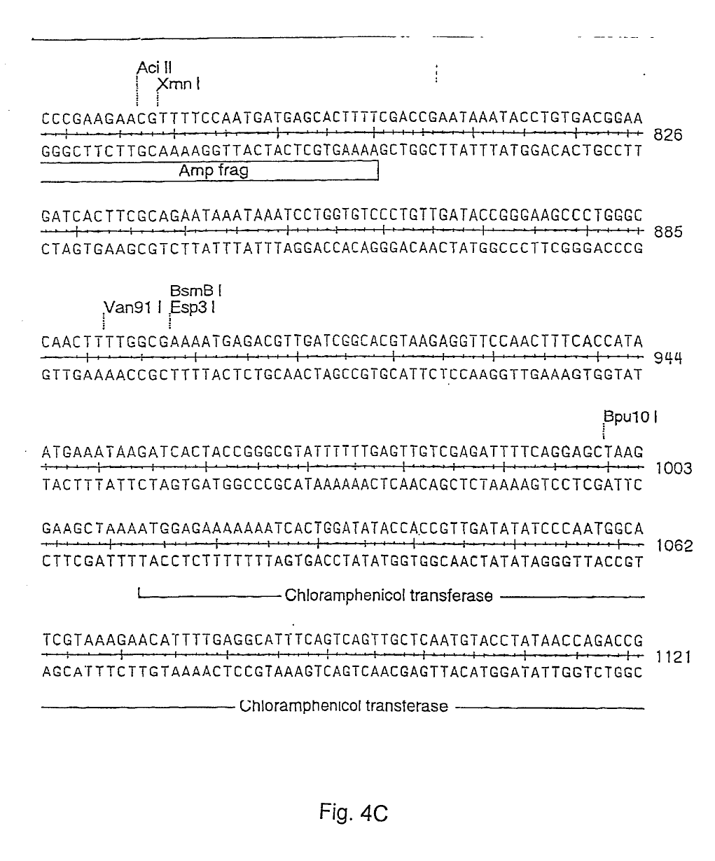

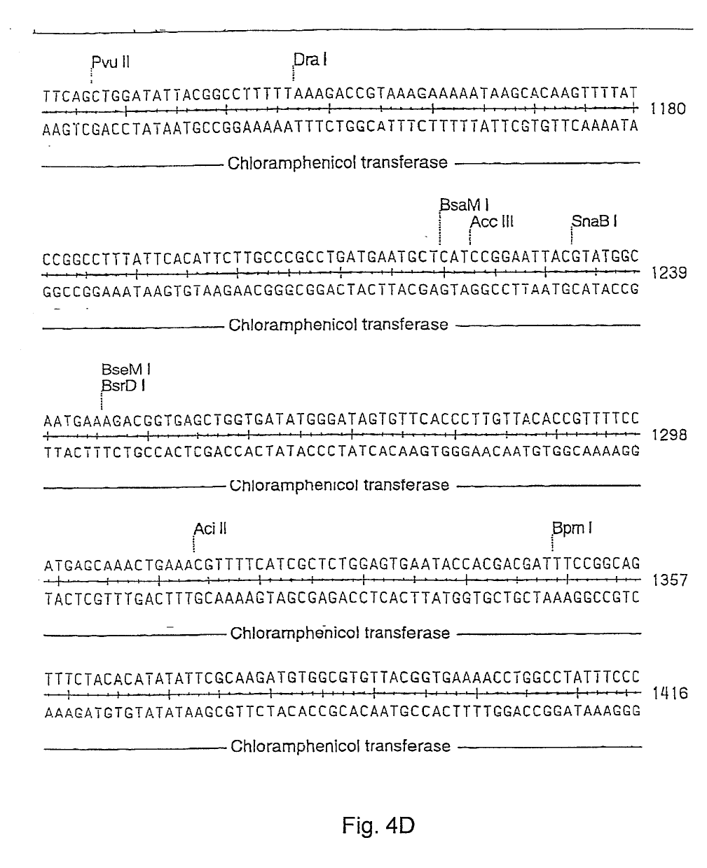

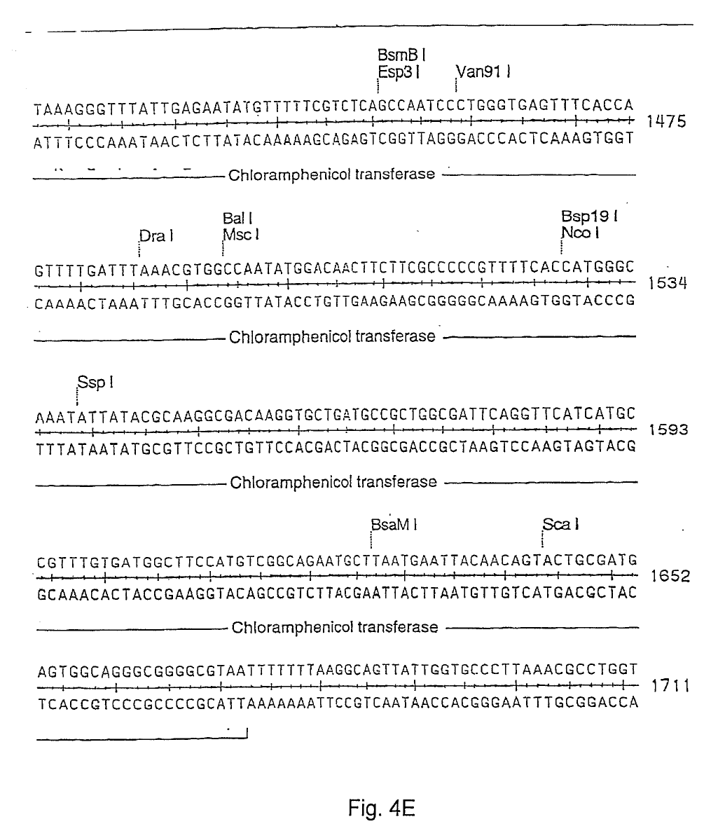

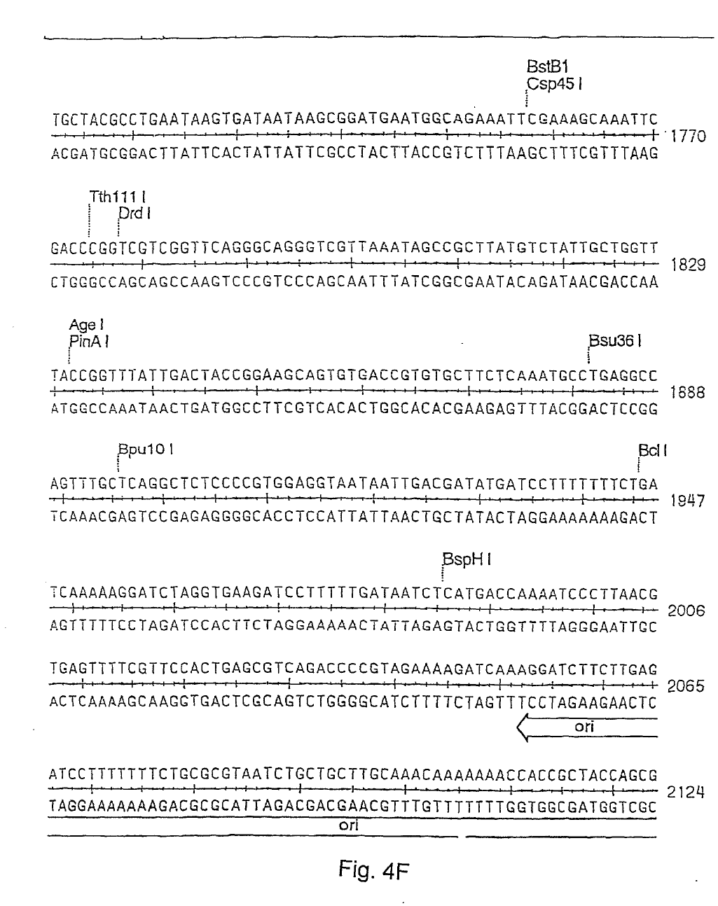

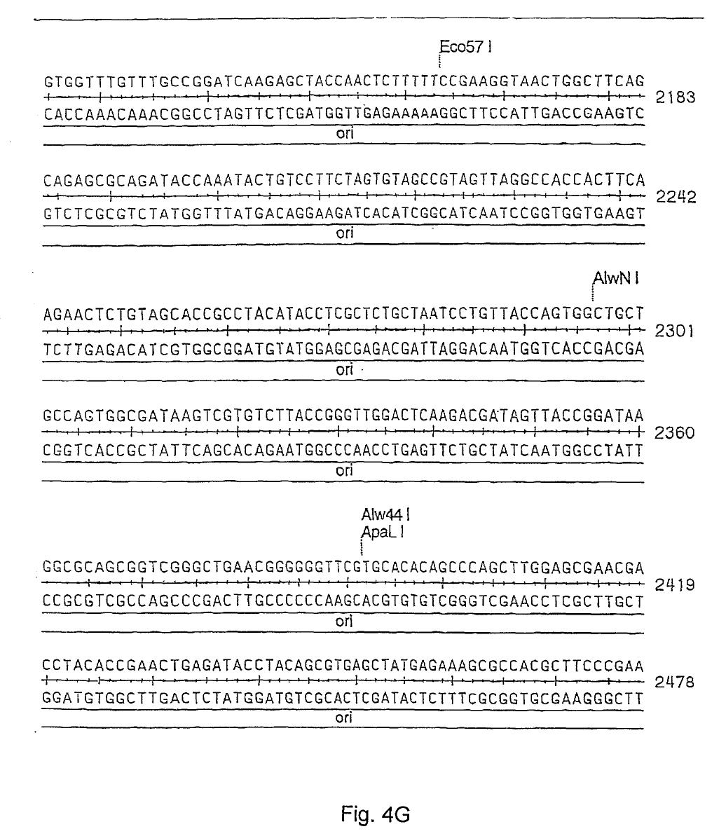

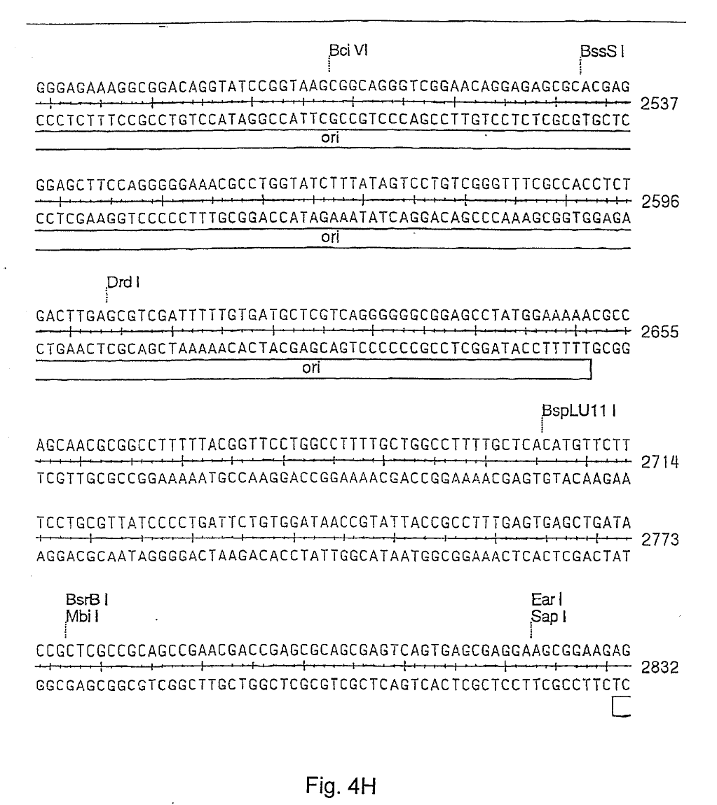

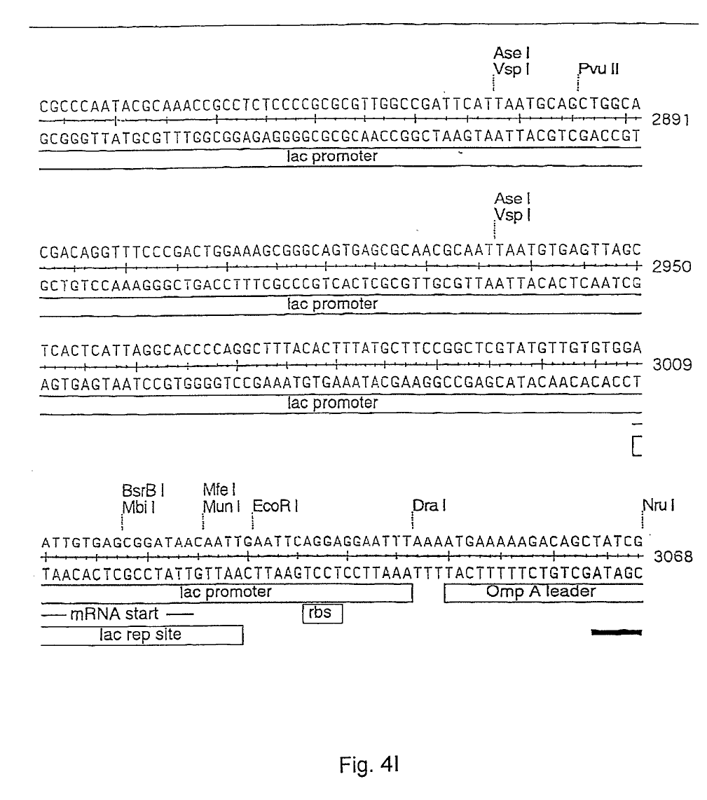

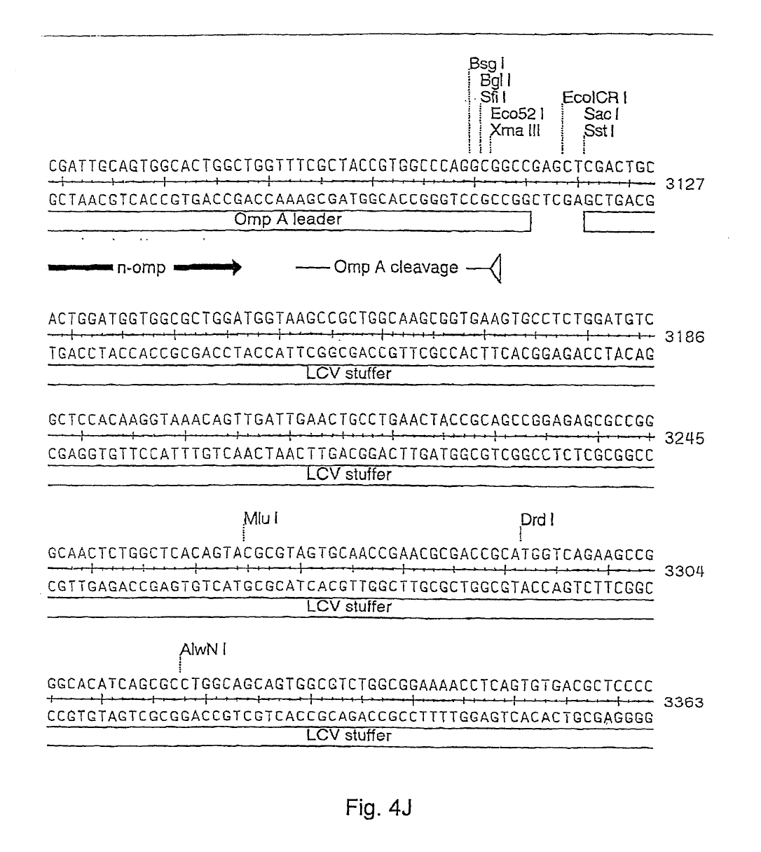

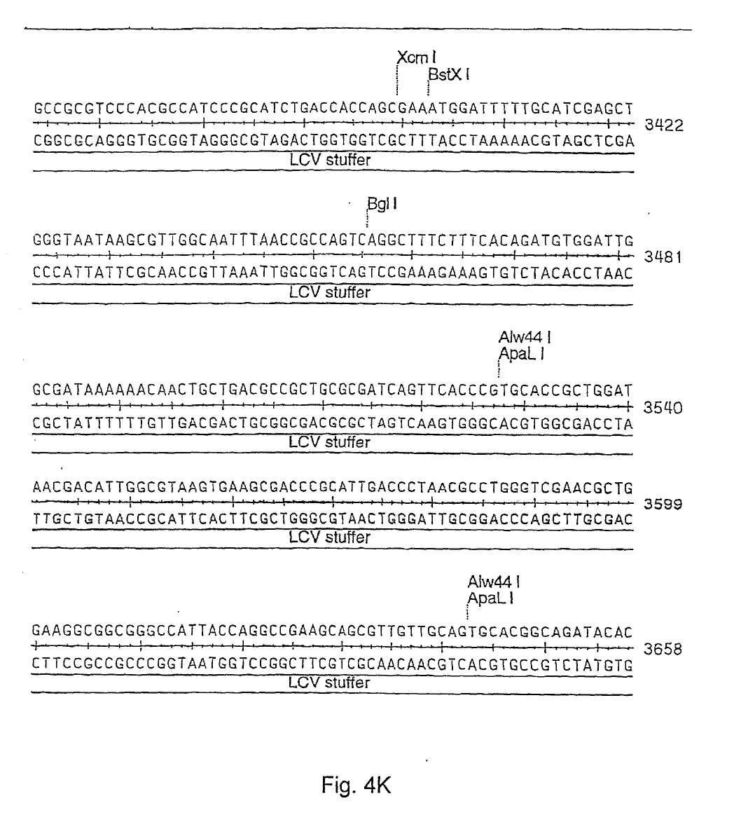

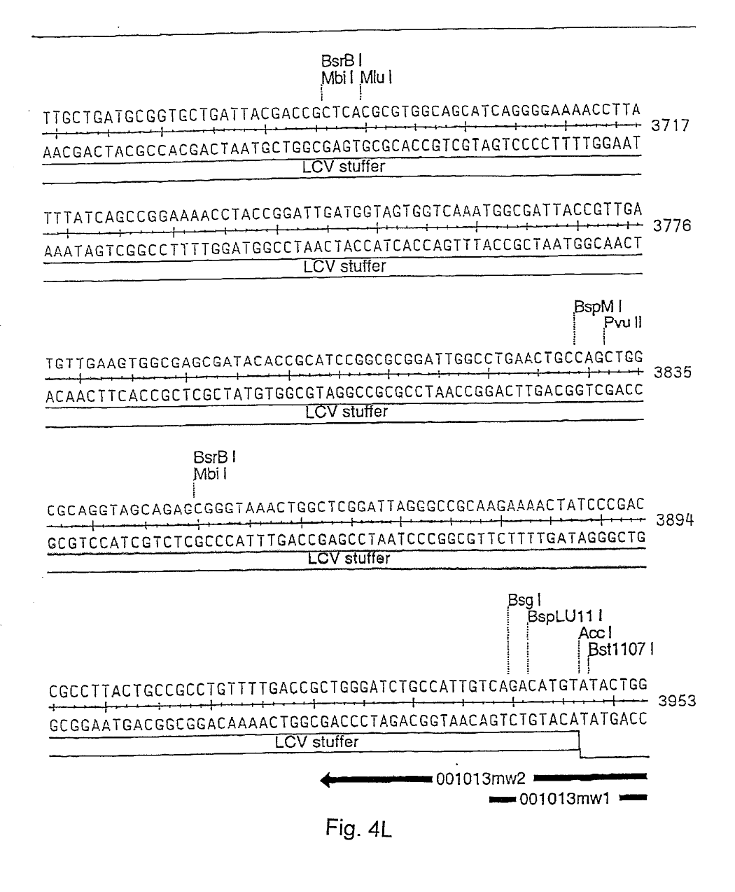

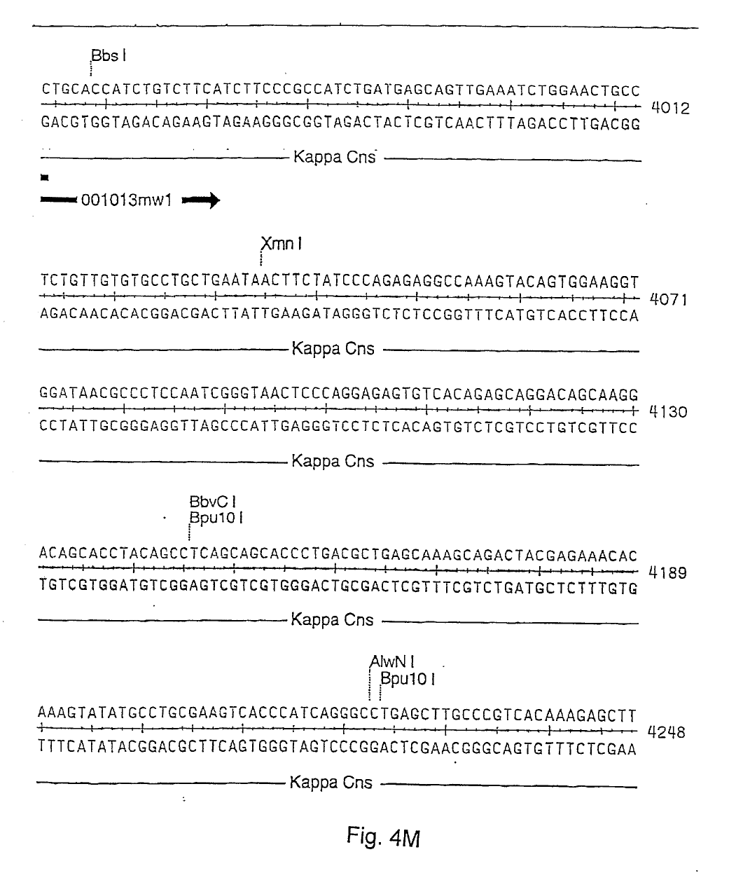

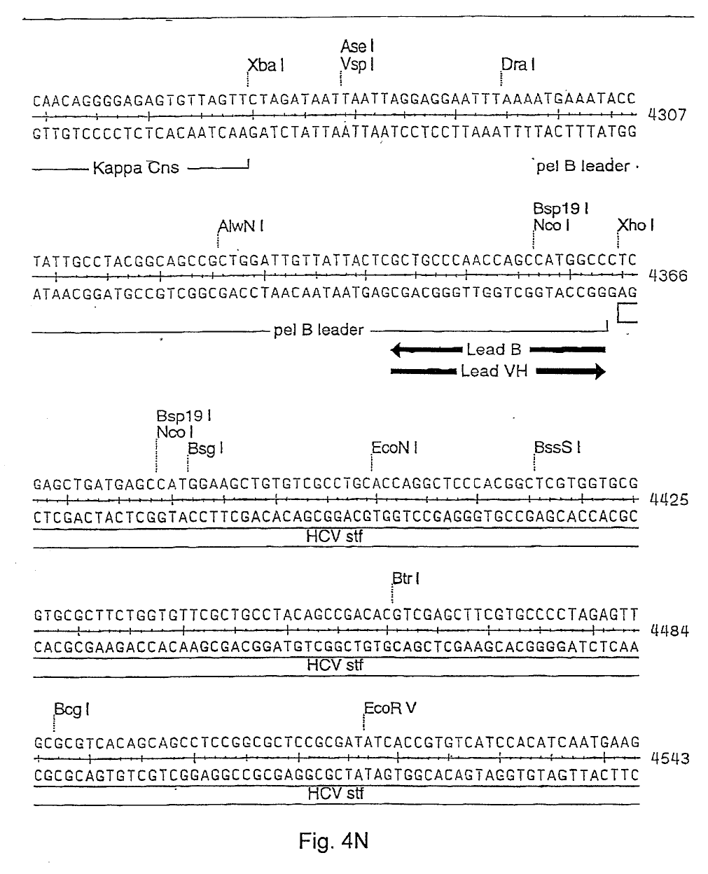

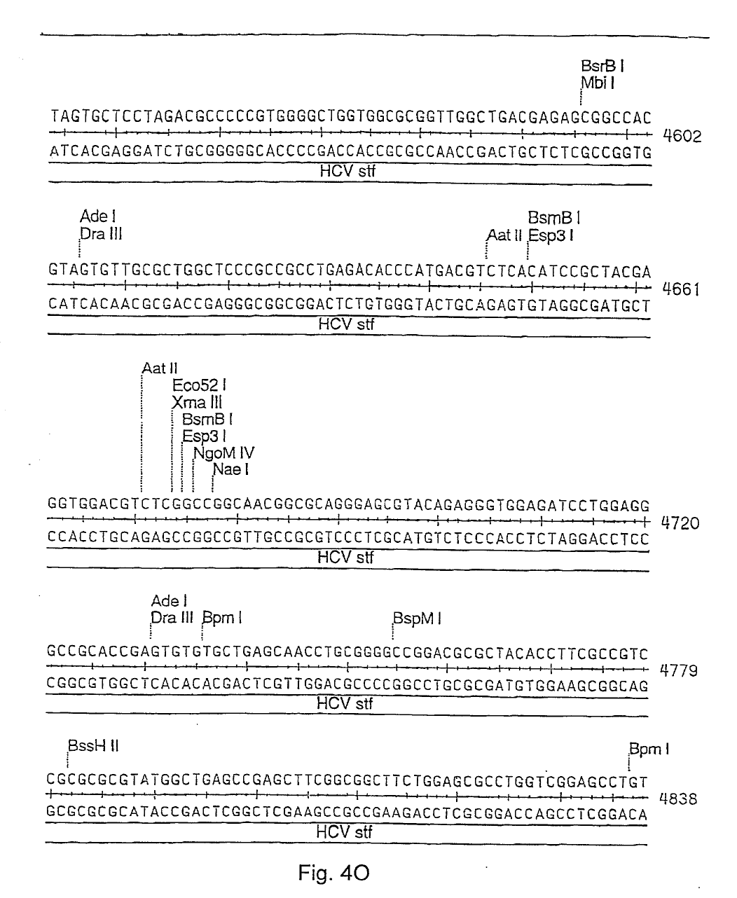

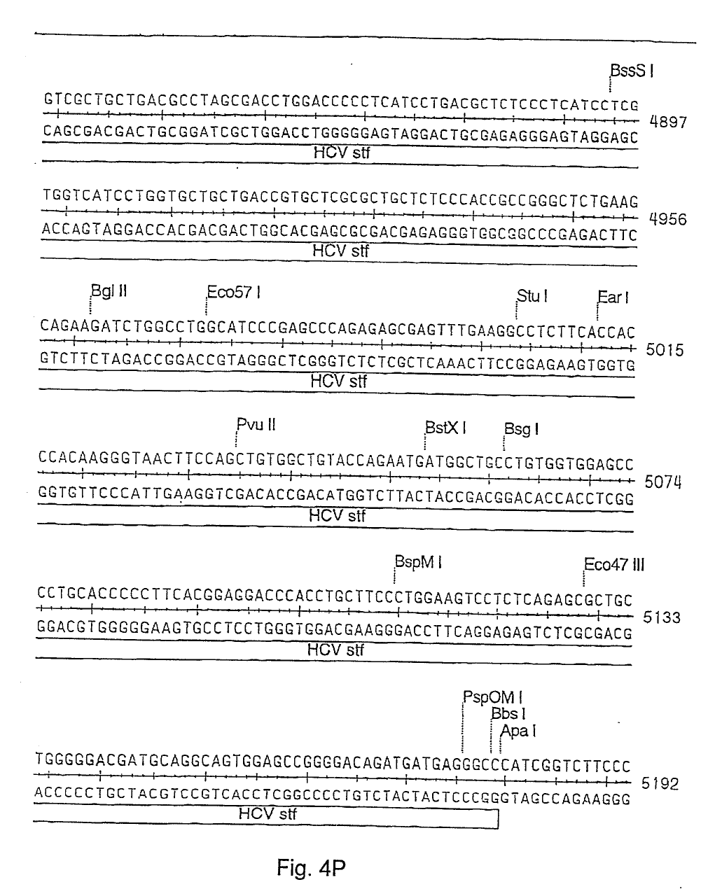

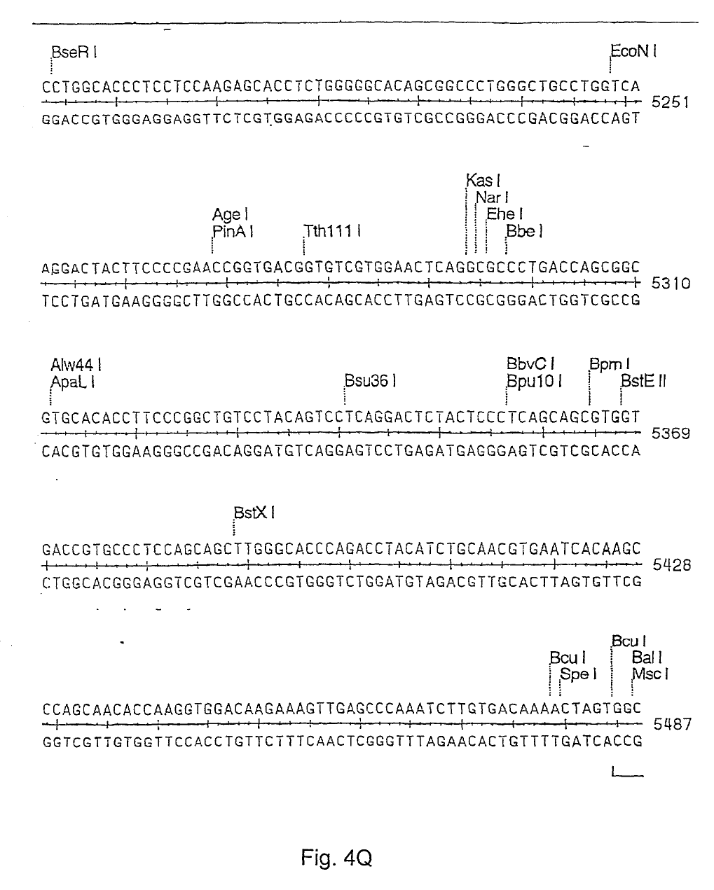

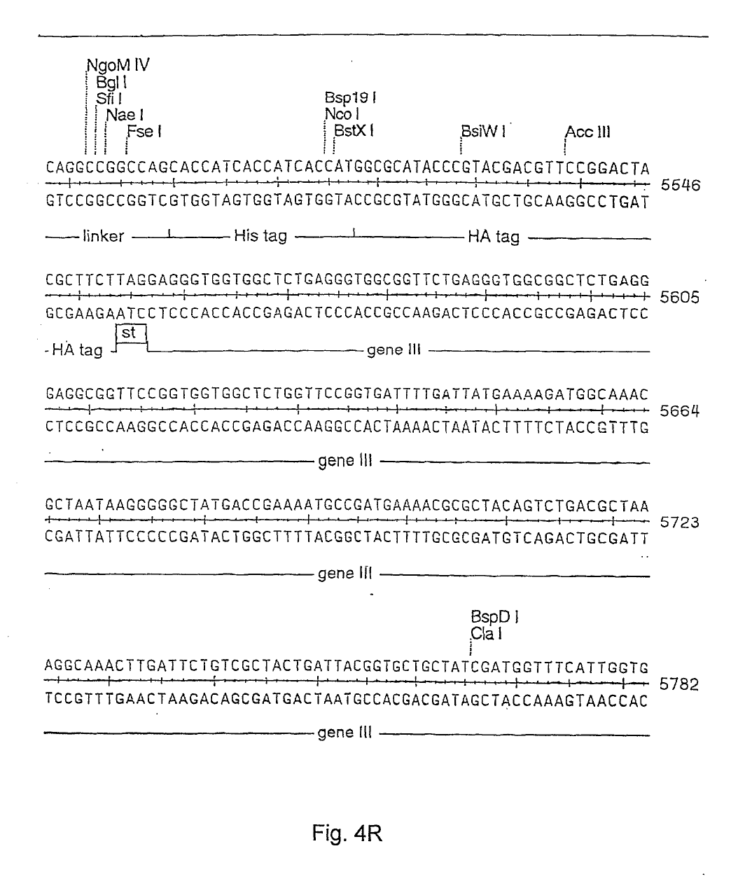

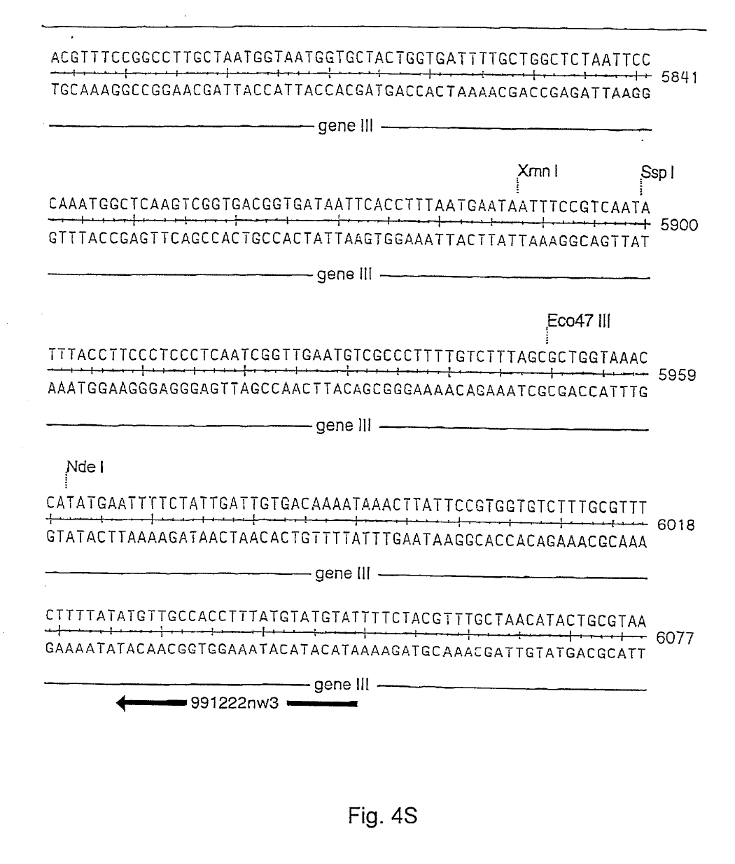

Any naturally occurring or synthetic plasmid can be used as the single strand, circular nucleic acid 12. Particularly useful plasmids include an origin of replication from filamentous phage (such as, for example an f1, M13 or fd origin of replication) which allows single stranded replication. Useful plasmids can contain from about 100 bases to about 10 kilobases (kb), more preferably from about 1700 bases to about 10 kb, most preferably, 1700 bases to about 7.5 kb. The single strand, circular nucleic acid 12 can be obtained from a double stranded plasmid, if desired, by methods known to those skilled in the art. Suitable starting plasmids are known and/or commercially available and include, for example, pComb 3H and its derivatives, Bluescript™ and its derivatives (available from Strategene, LaJolla, Calif.), M13 type vectors such as M13MP18 and M13MP19 (available from New England Biolabs, Beverly, Mass.), pHEN 1 (see Hoogenboom et al., 19917, Nucl. Acids Res. Vol. 19, pages 4133-4137), fd-tet-DOG 1 (id.), pGem plasmids (Promega, Madison, Wis.), pSL 1180 Superlinker Phagmid (available from Amersham Pharmacia, Piscataway, N.J.), pcDNA2.1 (available from Invitrogen, Carlsbad, Calif.), the pLITMUS Series, including pLITMUS 28, pLITMUS 29, pLITMUS 38, and pLITMUS 39 (available from New England Biolabs, Beverly, Mass.), and the pFLAG Series, including pFLAG-MAC, pFLAG-ATS, pFLAG-CTC, pFLAG-CTS and pFLAG-SHIFT (available from Sigma-Aldrich, St. Louis, Mo.). A particularly preferred starting plasmid is pRL5-CAT vector. pRL5-CAT is a derivative of pComb 3X (accessible in GenBank as accession no. AF268281) which has been modified to contain chloramphenicol resistance. A map and the sequence of the single strand pRL5-CAT vector are shown in FIGS. 2 and 3A-C, respectively. The double strand pRL5-CAT vector is shown in FIGS. 4A through 4T as including SEQ. ID. No: 1 and SEQ. ID. No: 2.

It is also contemplated that a linear nucleic acid strand with a downstream primer and an upstream collar sequence engineered onto the ends thereof can be used as the starting material in place of a circularized plasmid. The linear nucleic acid strand can be entirely or partially synthetic. Preferably, the linear nucleic acid strand includes an origin of replication which allows single strand replication.

The single strand, circular nucleic acid 12 is engineered to contain a downstream primer 14 adapted to anneal to a portion of messenger RNA encoding at least a portion of an antibody. The single strand, circular nucleic acid 12 is also engineered to include an upstream collar sequence 16 adapted to aneal to a portion of the messenger RNA encoding at least a portion of an antibody. The upstream collar sequence anneals to the mRNA at a position remote from (in the 5′ direction) the location at which the downstream primer anneals to the mRNA. Preferably, the upstream collar sequence anneals to a portion of the nucleic acid target that is at least about 20 nucleotides away from the portion of the target to which the downstream primer anneals. More preferably, there are about 25 to about 3000 nucleotides between the annealing sequences. Most preferably, the annealing sequences anneal to portions of the target that are separated by about 200 to about 2000 nucleotides.

The downstream primer and upstream collar sequence should be of sufficient length to support specific and stable hybridization to the target complementary mRNA. The annealing sequences may individually contain from about 10 nucleotides to about 50 or more nucleotides in length. Preferably, the individual annealing sequences are 15 to 35 nucleotides in length. Precise complementarity to the mRNA is not required. Mismatches are tolerable provided they do not interfere with initiation or termination of reverse transcription.

As those skilled in the art will readily appreciate, once the mRNA encoding any given antibody is isolated, it is a routine task to ascertain the nucleotide sequence of the DNA corresponding to the mRNA. In fact, the nucleotide sequence for the mRNA of various antibodies are well known and have been reported in the literature. See, for example, Kabat's Sequences of Proteins of Immunological Interest, 1991, 5^{th} Ed. NIH Publication 91-3242 and the publicly available VBase database (www.mrc-cpe.cam.ac.uk/imt-doc/) which is a comprehensive directory of sequences compiled from over a thousand published sequences including those in the current releases of the Genbank and EMBL data libraries. It is also a routine task to determine the structure of a suitable annealing sequence for any selected location along the mRNA once the sequence thereof is ascertained.

Advantageously, the available sequences for antibodies have been pooled and families of antibodies identified. Sequences have been determined, for example, for the Framework 1 (FR1) region of many antibodies within a family of antibodies. These sequences contain much in common for a given family of antibodies. Thus, a single primer may exhibit sufficient complementarity to adequately anneal to mRNA encoding a repertoire of antibodies within a family of antibodies.

Another source of information for designing annealing sequences suitable for use in the present methods is published primer sequences that have been used for polymerase chain reaction (PCR) amplification of double stranded nucleic acid. It is within the purview of those skilled in the art given the primer sequences employed on sense and antisense strands for PCR to easily design two sequences adapted to anneal to a single nucleic acid strand for use in accordance with the methods described herein. This is true whether the published primers are for PCR being accomplished on dsDNA encoding an antibody or related family of antibodies or dsDNA encoding any other polypeptide or related family of polypeptides.

The mRNA between the first and second locations of annealing determines the structure of the complementary nucleic acid sequence to be incorporated into the plasmid. Thus, for example, where the portion of the antibody coded by the nucleic acid sequence to be incorporated directly into the plasmid is the light chain, the downstream primer preferably anneals to the light chain constant region of the mRNA and the upstream collar sequence anneals to the light chain framework 1 (FR1) region of the mRNA. As another example, where the portion of the antibody encoded by the nucleic acid sequence to be incorporated directly into the plasmid is the heavy chain, the downstream primer preferably anneals to the heavy chain constant region of the mRNA and the upstream collar sequence anneals to the heavy chain framework 1 (FR1) region of the mRNA. If it is desired to directly incorporate a nucleic acid sequence encoding a smaller fragment of the antibody, say a specific complementarity determining region (CDR), the two annealing sequences are chosen that anneal to the portions of the mRNA encoding for the framework regions on either side of the desired CDR. From the foregoing description, it will be apparent to those skilled in the art how to select appropriate annealing sequences to incorporate a nucleic acid sequence encoding any desired portion of an antibody directly into the plasmid. Further illustrative, non-limiting examples using mRNA as a template are presented in Table 1. Those skilled in the art will envision from the examples provided the suitable annealing locations when other templates (such as, for example, first strand cDNA) are used.

TABLE 1

| Upstream collar | Downstream | Nucleic acid |
|---|---|---|
| sequence anneals | primer anneals to | generated in situ |
| to portion of mRNA | portion of mRNA | within plasmid |
| encoding . . . | encoding . . . | encodes for . . . |
| Light Chain | Light Chain | Entire light chain |
| Leader Region | Constant Region | |
| Light Chain | Light Chain | Entire light chain |
| FR1 Region | Constant Region | |
| Light Chain | Light Chain | Light Chain portion including |
| FR2 Region | Constant Region | CDR2, CDR3 Region |
| Light Chain | Light Chain | Light Chain portion including |
| FR3 Region | Constant Region | CDR3 Region |
| Light Chain | Light Chain | Entire light chain |
| Leader Region | FR4 Region | variable region |
| Light Chain | Light Chain | Entire light chain |
| FR1 Region | FR4 Region | variable region |
| Light Chain | Light Chain | Light Chain portion including |
| FR2 Region | FR4 Region | CDR2, CDR3 Region |
| Light Chain | Light Chain | Light Chain portion including |
| FR3 Region | FR4 Region | CDR3 Region |
| Light Chain | Light Chain | Light Chain portion including |
| Leader Region | FR3 Region | CDR1, CDR2 Region |
| Light Chain | Light Chain | Light Chain portion including |
| FR1 Region | FR3 Region | CDR1, CDR2 Region |
| Light Chain | Light Chain | Light Chain portion including |
| FR2 Region | FR3 Region | CDR2 Region |
| Light Chain | Light Chain | Light Chain portion including |
| Leader Region | FR2 Region | CDR1 Region |
| Light Chain | Light Chain | Light Chain portion including |
| FR1 Region | FR2 Region | CDR1 Region |
| Heavy Chain | Heavy Chain | Entire Heavy chain |
| Leader Region | Constant Region | |
| Heavy Chain | Heavy Chain | Entire Heavy chain |
| FR1 Region | Constant Region | |
| Heavy Chain | Heavy Chain | Heavy Chain portion including |
| FR2 Region | Constant Region | CDR2, CDR3 Region |
| Heavy Chain | Heavy Chain | Heavy Chain portion including |
| FR3 Region | Constant Region | CDR3 Region |
| Heavy Chain | Heavy Chain | Heavy Chain portion including |
| Leader Region | FR3 Region | CDR1, CDR2 Region |
| Heavy Chain | Heavy Chain | Heavy Chain portion including |
| FR1 Region | FR3 Region | CDR1, CDR2 Region |
| Heavy Chain | Heavy Chain | Heavy Chain portion including |
| FR2 Region | FR3 Region | CDR2 Region |
| Heavy Chain | Heavy Chain | Heavy Chain portion including |
| Leader Region | FR2 Region | CDR1 Region |
| Heavy Chain | Heavy Chain | Heavy Chain portion including |
| FR1 Region | FR2 Region | CDR1 Region |

At least one restriction site is located between the two annealing sequences on the engineered plasmid. The restriction site provides a location for cleaving the single strand, circular nucleic acid 12 to open the engineered plasmid and thereby make the annealing sequences available for annealing to mRNA. The sequences of many restriction sites are known to those skilled in the art as are the restriction enzymes which act to cleave a nucleic acid at a given restriction site. In one embodiment, a restriction site is chosen such that a similar restriction site does not appear elsewhere in the engineered plasmid. This ensures that cleaving occurs only at the desired site, that is, between the template annealing sequences.

In another embodiment, an oligonucleotide is hybridized to the restriction site. This increases the efficiency of digestion by the restriction enzyme at the double stranded site compared to single stranded restriction sites of the same type. This is an alternative method of ensuring that cleaving occurs only at the desired site, namely, between the template annealing sequences. Any well known endonuclease recognition sequence site is suitable for use herein. Depending on the plasmid employed as the starting material, suitable restriction sites may include Pvu II, Sma I, Hinc II, Hind III and combinations thereof. When possible, template annealing sequences are chosen which when placed adjacent to one another in the plasmid define a restriction site. Thus, no structure in addition to the template annealing sequences may be necessary to form the restriction site. It should, of course be understood that nucleotides in addition to those contained in the downstream primer or upstream collar sequences can be engineered into the plasmid between the template annealing sequences to form the restriction site provided that the additional nucleotides do not interfere with the reverse transcription process or adversely affect the performance of the antibody or antibody fragment encoded by the nucleic acid sequence to be incorporated directly into the plasmid.

In an alternative embodiment shown schematically in FIG. 1B, two restriction sites 15a, 15b which can be the same or different are located between the template annealing sequences 14, 16. Upon cleaving (as described hereinafter in more detail) a cleaved plasmid is produced that is a single, linear nucleic acid strand having the downstream primer and upstream collar sequences at the ends thereof. The sequence 17 located between restriction sites 15a, 15b is essentially discarded upon cleaving. This embodiment may be employed to simplify the design process for the engineered plasmid.

It is also contemplated that two sets of template annealing sequences can be engineered into a single plasmid with restriction sites located between each set of annealing sequences. In this manner, the same engineered plasmid can, for example, be used to directly incorporate either of two portions of complementary nucleic acid into the plasmid, depending upon which of the two restriction sites is cleaved. Selectivity in cleaving can be attained by hybridizing a suitable oligonucleotide to one of the two restriction sites to provide a double strand at that location thereby increasing the effectiveness of restriction enzyme at the selected restriction site.

Once the desired sequence for the downstream primer and upstream collar sequences is established, the sequence can be provided to a commercially available service that produces an insert having the desired sequence by building it nucleotide by nucleotide using techniques that are well known to those skilled in the art. Suitable commercially available services include those provided by Sigma Genosys, Woodland, Tex. and Operon Technologies, Alameda, Calif.. Annealing oligonucleotide sequences can be synthesized using established oligonucleotide synthesis methods. Methods to produce or synthesize oligonucleotides are well known in the art. Such methods can range from standard enzymatic digestion followed by nucleotide fragment isolation (see for example, Sambrook et al, Molecular Cloning: A Laboratory Manual, 2^{nd} Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method).

Techniques for engineering the template annealing sequences and restriction site into the plasmid are well known to those skilled in the art. Typically, the starting plasmid is cleaved by a suitable restriction enzyme. The sequence to be inserted is then ligated into the plasmid. See, for example, Molecular Cloning—A Laboratory Manual, 2^{nd} Ed., Sambrook et al., Cold Spring Harbor Laboratory Press, 1989. As those skilled in the art will appreciate, an insert containing the two annealing sequences and restriction site(s) can be combined with a substantially complementary oligonucleotide to provide a strand with “sticky ends” that can be readily ligated into the starting plasmid using well known techniques. Either strand can then be rescued using known methods for use in the methods described herein.

The engineered plasmid can be transformed into a suitable host cell i.e., prokaryotes and eukaryotes, such as yeast, bacteria, insect cells, etc. (e.g., E. coli, CHO cells) to be amplified. Any suitable technique (e.g., electroporation) known to those skilled in the art can be used to transform the cell with the engineered plasmid.

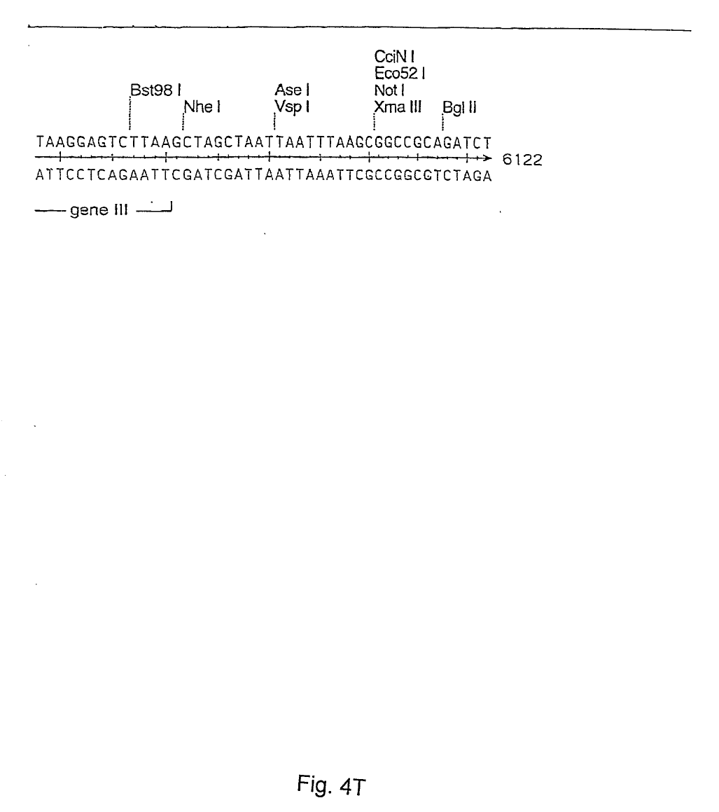

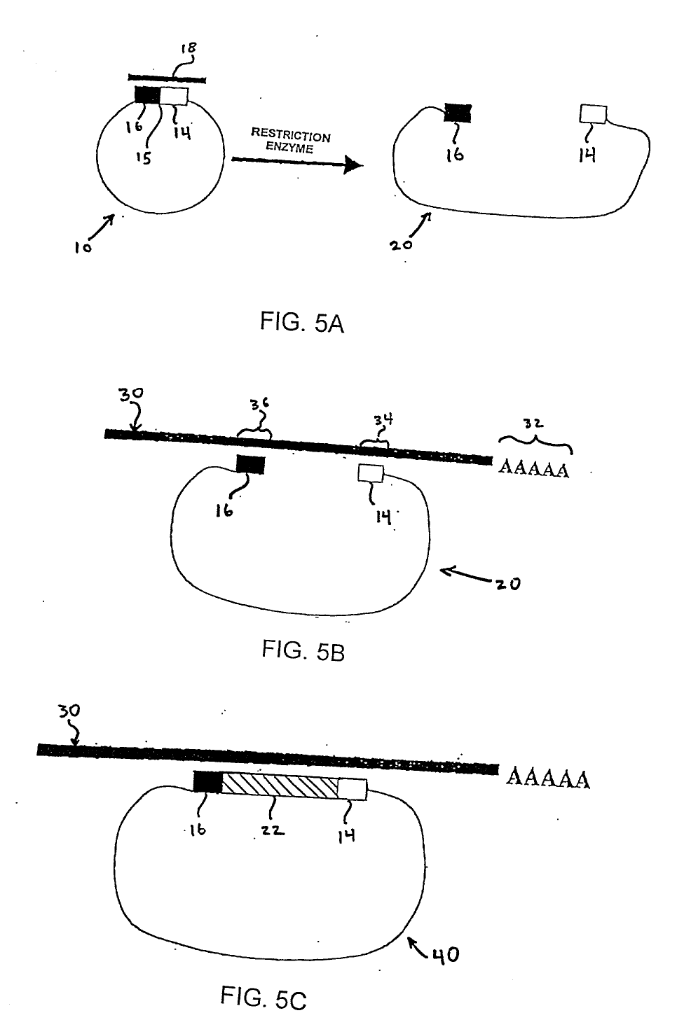

As mentioned above, the engineered plasmid is cleaved to open the plasmid and thereby make the downstream primer and upstream collar sequences available for annealing to mRNA. This step of the process is schematically represented in FIG. 5A. To cleave the engineered plasmid 10, an oligonucleotide 18 adapted to hybridize at the restriction site can be provided. The oligonucleotide 18 creates a double stranded portion on the engineered plasmid 10 to facilitate and improve the efficiency of the cleavage at that site. Providing an oligonucleotide to create a double stranded portion on the engineered plasmid is particularly preferred where more than one restriction site of the same type is present on the engineered plasmid. As those skilled in the art will also appreciate, certain restriction enzymes will not cleave a single strand but require a double strand to function. In those instances, hybridization with an oligonucleotides must be performed. The structure of the oligonucleotide is complementary to and determined by the sequence of nucleotides flanking the restriction site. Once the desired sequence for the oligonucleotide is established, the sequence can be provided to a commercially available service that produces an oligonucleotide having the desired sequence by building it nucleotide by nucleotide using techniques that are well known to those skilled in the art. After the oligonucleotide has hybridized to the engineered plasmid, exposure to a restriction enzyme cleaves engineered plasmid 10 between template annealing sequences 14 and 16 to form cleaved plasmid 20 (see FIG. 5A).

The cleaved plasmid can then be exposed to mRNA encoding at least a part of an antibody in the presence of reverse transcriptase and nucleotides under conditions that allow reverse transcription to proceed.

Messenger RNA encoding at least part of an antibody can be obtained from any tissue containing antibody producing cells. Such cells include, for example, spleen cells, peripheral blood cells, lymph nodes, inflammatory tissue cells and bone marrow cells. The antibody-producing cells can be of human or non-human origin. mRNA can be obtained directly from the tissue (i.e., without previous treatment to remove cells which do not produce antibody) or can be obtained after the tissue has been treated to increase concentration of antibody-producing cells or to select a particular type(s) of antibody-producing cells (i.e., treated to enrich the content of antibody-producing cells). Antibody-producing cells can be stimulated if desired by an agent which stimulates antibody mRNA production (e.g., lipopolysaccharide). Alternatively, antibody producing cells can be stimulated in vitro using antigens. Also, agents that stimulate innate and adaptive immune systems can be simultaneously used. Illustrative sources of cells from which mRNA can be collected include naïve humans, antigen-challenged humans (e.g., humans exposed to antigens intentionally or in the environment or suffering or recovering from disease), naïve animals, antigen-challenged animals and grafted animals.

The cleaved plasmid 20 can then anneal to mRNA 30 as schematically shown in FIG. 5B. As those skilled in the art will appreciate, mRNA encoding an antibody typically has a poly-A portion 32 at the 3′ end thereof. The portion of the mRNA to which the downstream primer 14 anneals is indicated by the numeral 34 in FIG. 5B and the portion of the mRNA to which the upstream collar sequence 16 anneals is indicated by the numeral 36 in FIG. 5B. The selection of portions 34 and 36 will depend, as discussed above, on the specific nucleic acid that is to be incorporated directly into the plasmid.

Direct incorporation of a desired nucleic acid encoding at least a part of an antibody is then achieved by reverse transcription. The conditions under which reverse transcription occurs are well understood by those skilled in the art. To achieve reverse transcription it is necessary that reverse transcriptase and a supply of nucleotides be provided in an aqueous solution containing the mRNA and the cleaved plasmid. The reverse transcriptase employed can be selected, for example, from those commercially available reverse transcriptases including Superscript M-MLV RT (Life Technologies, Gaithersburg, Md.), AMV-RT (Roche Molecular Biochemicals, Indianapolis, Ind.) Delta Tth polymerase (Toyono Ltd., Osaka, Japan), Tth (Perkin-Elmer, Foster City, Calif.). The foregoing commercially available reverse transcriptases are provided as a kit that includes suitable buffers and nucleotides as well as detailed instructions with respect to reaction conditions under which reverse transcription can be achieved. Typically, the temperature used for the reverse transcription reactions will normally be selected in the range from between 30° and 75° to provide the optimal activity of the reverse transcriptase employed.

Generally speaking, reverse transcription will proceed along the mRNA synthesizing the newly formed cDNA in the 5′ to 3′ direction to fill in the gap in the cleaved plasmid 20 created between the downstream primer 14 and upstream collar sequence 16 with nucleic acid 22 as shown schematically in FIG. 5C to form what will hereinafter be referred to as plasmid vector 40. The nucleic acid 22 incorporated directly into the plasmid will be complementary to the mRNA between portions 34 and 36. The nucleic acid sequence incorporated directly into the plasmid is thus essentially first strand complementary DNA (cDNA). In alternative embodiments, for example where first strand cDNA is used as a template, the nucleic acid sequence incorporated directly into the plasmid is second strand cDNA. In such embodiments, a polymerase (rather than a reverse transcriptase) should be employed. Suitable polymerases include but are not limited to Klenow, T4 DNApol, T7 DNApol, Taq1, Vent, Deep Vent, Pwo and Pfu, each of which is well known to those skilled in the art.

The cDNA is formed in situ within the cleaved plasmid. That is, in contrast to processes wherein the cDNA is produced separately from the plasmid and then simply ligated intact into the plasmid. The present process generates the cDNA directly within the cleaved plasmid nucleotide by nucleotide by the process of reverse transcription.

A ligase can be employed to seal the nick between the newly synthesized nucleic acid and the upstream collar sequence. A suitable ligase known to those skilled in the art is T4 DNA ligase, which is commercially available from New England Biolabs, Beverly, Mass. In alternative embodiments where DNA is used as the template for generating a nucleic acid in situ, suitable ligases include, but are not limited to T4 DNA ligase or DNA ligase (E. coli, NAD), or Taq DNA ligase.

Depending on the particular reverse transcriptase employed and the buffer system provided therewith, an adjustment to the buffer may be required to ensure ligase activity. In an alternative embodiment, the mRNA and plasmid vector can be transformed into a host cell which affects ligation.

In an alternative embodiment, the use of a collar sequence is eliminated. In this embodiment, the template is cleaved at a site adjacent the 3′ end of the nucleic acid sequence to be incorporated into the plasmid. A hybridizing oligo may advantageously be used during this cleavage to convert the restriction site in the first strand cDNA to double stranded DNA for specific restriction enzyme digestion. Second strand synthesis is achieved by hybridizing the primer sequence of the single stranded plasmid to the cleaved template and synthesizing the second strand cDNA until it reaches the end of the cleaved 1^{st} strand cDNA template. After second strand synthesis is complete, the free end of the 2^{nd} strand cDNA is ligated to the free end of the single stranded vector using any known technique. Preferably, a bridging oligo is employed which hybridizes to and holds the two free ends together, thereby facilitating ligation. The ligated single stranded vector with the incorporated 2^{nd} strand cDNA is then transformed into a host cell, as detailed above in connection with previous embodiments.

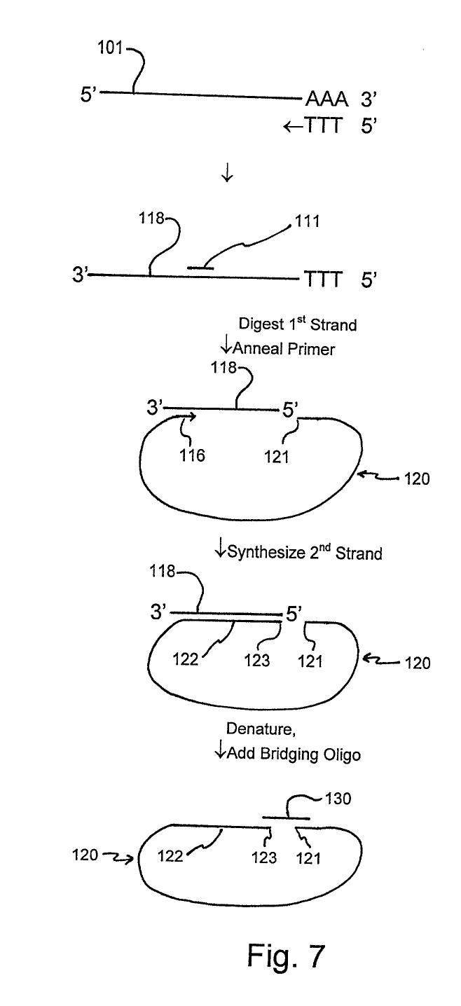

FIG. 7 is a flow-chart showing the steps in an illustrative process of this embodiment. In this example, first strand cDNA encoding an antibody is used as the template. The first strand cDNA is produced using conventional methods from mRNA 101 using oligo dT. The first strand cDNA is then specifically cleaved at a specific site within the constant region. Examples of such specific sites are, but not limited to: Apa L1 or Alw44 l for the IgG HC, Dra III for the IgM HC, Sac 1 for the Kappa LC, and Sma1 for the Lambda LC. A hybridizing oligo 111 converts the restriction site in the first strand cDNA to double stranded DNA for specific restriction enzyme digestion. Second strand synthesis occur by hybridizing the primer sequence 116 (such as, for example the FR1 sequence) to the cleaved first strand cDNA template. Second strand cDNA synthesis proceeds along the cleaved first strand cDNA template until the end of the cleaved 1^{st} strand cDNA template is reached. After polymerization of the newly synthesized second strand 122 is complete, the cleaved first strand cDNA template is removed by any known technique such as, for example, heat denaturation. The free end 123 of the newly synthesized 2^{nd} strand cDNA is then ligated to the free end 121 (e.g., constant region end) of the single stranded vector 120 using any known technique. For example, a bridging oligo 130 can be used to hybridize to and hold the two free ends 121, 122 together, thereby facilitating ligation. The resulting single stranded vector 120 contains the remaining constant region sequences encoded by the first strand cDNA down stream of the restriction site. The ligated single stranded vector with the incorporated 2^{nd} strand cDNA can then be transformed into a suitable host cell such as, for example, bacteria.

Since the FR1 regions on mRNA encoding antibodies contain variations, a set of FR1 annealing sequences would be necessary if it is desired to capture a repertoire of antibody gene messages for both light and heavy chains. Incorporation of multiple FR1 annealing sequences into individual plasmids would create a panel of unique vectors. By performing multiple reverse transcription reactions employing the various engineered plasmids with mRNA and then pooling the results, a light or heavy chain library of antibodies can be created. If nucleic acid sequences encoding light and heavy chains are incorporated directly into separate plasmid vectors specific for each, the nucleic acid sequences can be isolated and combined into a single plasmid vector to form Fab antibody libraries in a subsequent cloning step. Alternatively, the light and then heavy chains could be reverse transcribed sequentially into a single pool of vectors.

Once formed, the plasmid vector can be processed in a variety of ways. For example, the plasmid vector can be transformed into a host cell (e.g. E. coli) using any of the techniques known to those skilled in the art (e.g., electroporation). Alternatively, the second strand (in whole or in part) can be synthesized in vitro and then can be isolated from the plasmid vector by appropriate digestion. The cDNA recovered can be manipulated in any desired manner using known techniques.

As those skilled in the art will readily appreciate, the nucleic acid encoding at least a portion of an antibody that has been formed in situ within a plasmid in accordance with this disclosure can also be used to generate mRNA (or fragments thereof) that encode for an antibody (or fragment thereof) as a vehicle for producing antibodies or antibody fragments.

The following example is included for purposes of illustration and should not be construed as limiting the subject matter of the present specification and/or claims.

### EXAMPLE 1

The following example shows the direct incorporation of antibody genes to form a plasmid vector by reverse transcription of messenger RNA encoding Fab of antibody molecules in the Vkappa 1 and VH1 families. In summary, a starting plasmid is engineered to contain two annealing sequences that are complementary to antibody mRNA. The upstream collar oligonucleotide sequence was designed to anneal to a family of framework (FR)1 regions and the downstream primer sequence was designed to anneal to the constant region domain. The two annealing sequences were engineered into the plasmid in tandem. Once the engineered plasmid was prepared the two annealing sequences were separated by a restriction digest to produce a cleaved plasmid. The portion of the cleaved plasmid that anneals to the antibody constant region serves as a primer for reverse transcriptase to generate first strand cDNA. Transcription is terminated when the transcriptase encounters the upstream collar sequence of the cleaved plasmid in the antibody FR1 region. Ligation of the product then generates a plasmid vector containing a nucleic acid encoding an antibody light or heavy chain sequence. The plasmid vector is transformed into E.coli, where it is converted to double stranded DNA plasmids and amplified.

Construction of Engineered Plasmids

Kappa Light Chain Vector

Phagemid vector pRL5-CAT was modified between the Sac l and Xba I sites to contain the FR1 collar sequence:

5′ G GGT CAT CTG GAT GT (C/T) 3′ (SEQ. ID. NO: 3)

immediately adjacent to the Kappa constant region primer sequence:

5′ A TTA ACA CTC TCC CCT GTT GAA GCT CTT TGT GAC GGG CGA ACT CAG GCC C 3′ (SEQ. ID. NO: 4).

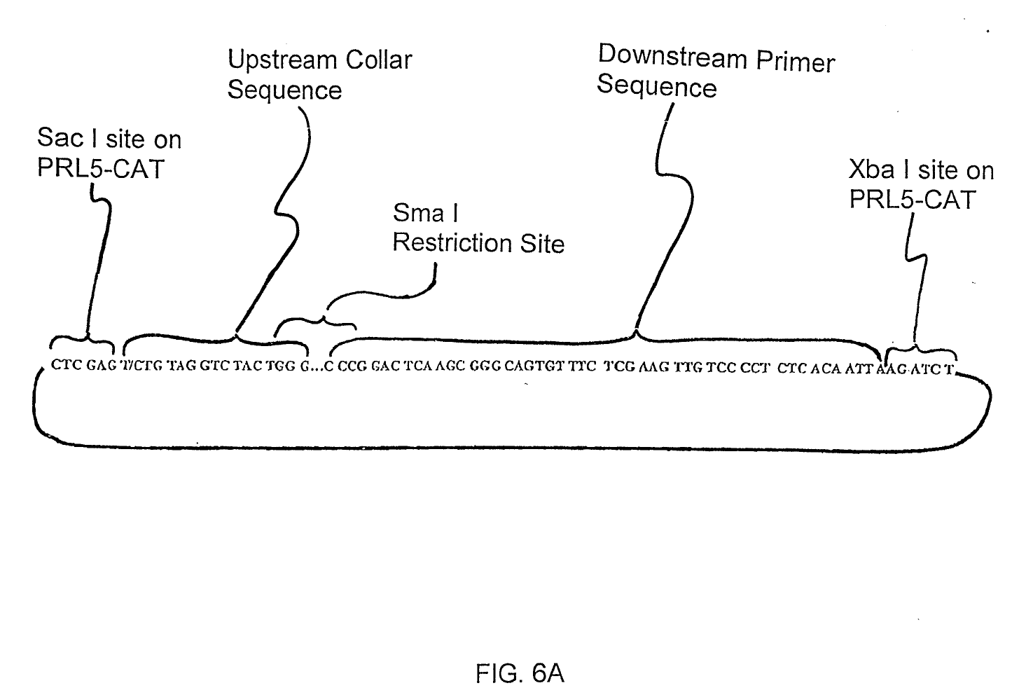

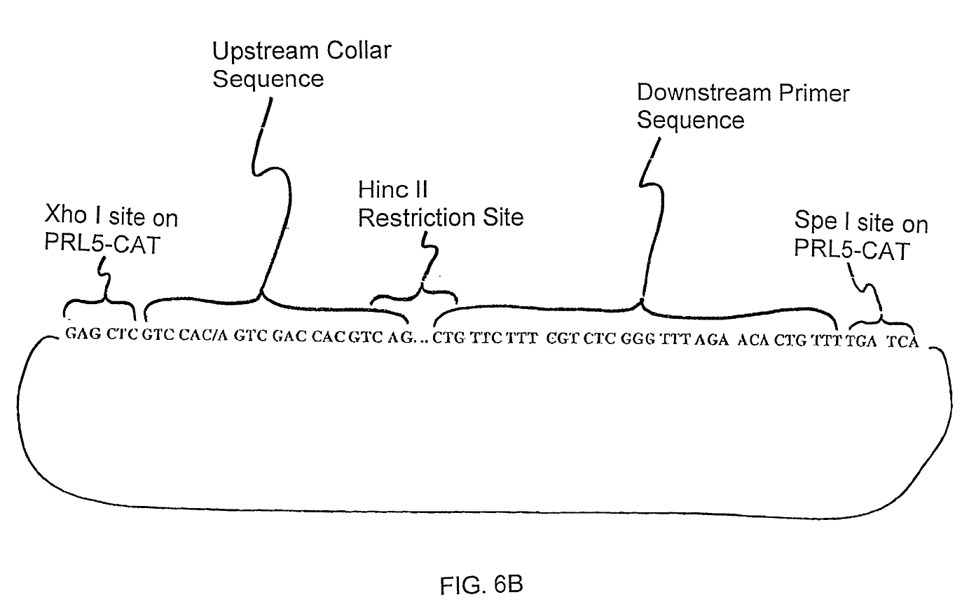

The FR1 collar sequence was designed based on information obtained from the VBase database. Essentially this sequence is the reverse complement of Vkappa 1A primers that have been slightly modified to add nucleotides to form a restriction site. The constant region primer sequence was designed based on information obtained from Kabat, supra. The junction of the two annealing sequences in this case forms a Sma I restriction site. (See FIG. 6A.)

The modifications to pRL5-CAT vector were accomplished by creating duplex oligonucleotide inserts with Sac I and Xba I compatible overhangs. For the Vkappa 1A annealing sequences identified above forming a Sma I restriction site, the complementary oligonucleotides are:

| | |
|---|---|
| 5′ C(A/G)AC ATC CAG ATG ACC C G GGC CTG AGT TCG CCC GTC ACA | (SEQ. ID. NO:5) |
| AAG AGC TTC AAC AGG GGA GAG TGT TAA TT 3′ | |

as the coding sequence and:

| | |
|---|---|
| 5′ CTA GAA TTA ACA CTC TCC CCT GTT GAA GCT CTT TGT GAC GGG | (SEQ. ID. NO:6) |
| CGA ACT CAG GCC CGG GTC ATC TGG ATG T(C,T)G AGC T 3′ | |

as the non-coding sequence. In each case the coding and non-coding oligonucleotides were mixed at an equal molar ratio and heat denatured at 70°. The samples were cooled slowly to allow duplexes to form. Duplexed oligonucleotides were used as insert in a ligation with the Sac I/Xba I digested pRL5-CAT vector. Following ligation, the engineered plasmid was electroporated into bacterial cells. Transformed bacterial clones were selected on agar plates containing antibiotic (choloramphenicol). DNA was prepared from individual bacterial colonies and then sequenced to verify incorporation of the correct FR1-Constant region annealing sequences. Due to the one degenerate position in the FR1 priming region, each vector strategy actually has two members (hence the designation (T/C) in SEQ. ID. Nos: 3 and 6). Equal amounts of engineered plasmid containing either the T or C in the first position past the Sac I site were combined to form the FR1 collar sequence.

Heavy Chain Vector

Phagemid vector pRL5-CAT was modified between the Xho I and Spe I sites to contain the FR1 collar sequence:

5′ GA CTG CAC CAG CTG (C/A)AC CTG 3′ (SEQ. ID. No: 7)

immediately adjacent to the heavy chain CH1 constant region primer sequence:

5′ TTT GTC ACA AGA TTT GGG CTC TGC TTT CTT GTC 3′ (SEQ. ID. NO: 8). The FR1 collar sequence was designed based on information obtained from the VBase database. Essentially, the FR1 collar sequence is a reverse complement of VH1A primers that have been slightly modified to add nucleotides to form a restriction site. The heavy chain CH1 constant region primer sequence was designed based on information contained in Kabat, supra. The junction of the two annealing sequences forms a Hinc II restriction site (see FIG. 6B). There are no other Hinc II sites in the pRL5 vector. As described above, the degenerate position in the FR1 will result in a mixture of two vectors differentiated by the FR1 collar sequence.

The heavy chain vector modifications were accomplished essentially as described above for the light chain. The complementary oligonucleotide sequences are:

| | | |
|---|---|---|
| 5′ TCG AGC AGG T(G/T)C AGC TGG TGC AGT CGA CAA GAA AGC AGA GCC | (SEQ. ID. NO:9) | / |
| CAA ATC TTG TGA CAA AA 3′ | | |

for the coding sequence and:

| | |
|---|---|
| 5′ CTA GTT TTG TCA CAA GAT TTG GGC TCT GCT TTC TTG TCG ACT GCA CCA | (SEQ. ID. NO:10) |
| GCT G(A,C)A CCT GC 3′ | |

for the non-coding strand. The duplexed oligonucleotides have overhangs compatible with Xho I and Spe I. The insert is ligated into Xho I/Spe I digested pRL5-CAT.

Helper Phage Rescue And Purification Of Single Stranded Plasmids

Because the engineered plasmid has a phage F1 origin, the dsDNA plasmid present in the bacterial cells can be recovered as ssDNA and packaged in a phage particle. After an overnight growth the phage particles were isolated and the encapsulated ssDNA released. Described here is the method used to rescue the ssDNA for the heavy chain vector, pRL5 CAT ssHC-VEC. Those skilled in the art will appreciate that the same protocol could be used to isolate ssDNA from the other vectors having an f1, M13 or fd origin of replication from filamentous phage.

Bacteria harboring the double stranded plasmid pRL5 CAT ssHC-VEC were grown at 37° C. in 100 ml of SB liquid media (containing 25 ug/ml chloramphenicol) to and OD 600 nm of '0.8 in a shaking flask. Helper phage are then added (1 ml of ˜1.1×10⁹ pfu/ml) and the culture grown for an addition two hours at 37° Shaking. Kanamycin was then added to a final concentration of 70 ug/ml and the culture allowed to grow overnight. The following morning the bacteria were spun down at 5000 rpm at 4° for 20 minutes in 500 ml bottles. The supernatant containing phage particles was then transferred to 50 ml tubes and spun harder (9000 rpm at 4° for 20 min.) to remove residual bacteria. The supernatant was then filtered through a 0.2 um filter. Phage particles were precipitated by adding 8 mls of 20% PEG/2.5M NaC1 to 32 mls of supernatant in a 40 ml screw top centrifuge tube. Tubes were mixed by inversion and placed on ice for 30 minutes then centrifuged for 20 minutes at 9000 rpm. The supernatant was poured off and the tube inverted for 10 minutes to allow the remaining PEG containing supernatant to drain from the phage pellet. Phage pellets were then resuspended in 2 mls total of TE and then filtered through a 0.2 um Minisart Plus® syringe tip filter (Sartorius, Germany). Phage particles were cracked open by adding an equivalent volume of equilibrated phenol, vortexing, centrifugation, and collection of the supernatant. The supernatant was extracted once more with phenol and then twice with phenol/chloroform/isoamyl alcohol. The supernatant was then heated in a 100° waterbath for 15 minutes, cooled to room temperature and then extracted 6 more times with phenol/chloroform/isoamyl alcohol. DNA was then precipitated by adding 1/10^{th} the volume of 3M sodium acetate and 2.5 times the volume of ethanol. DNA was precipitated for at least 2 hrs at −20° C., centrifuged, 70% ethanol washed twice, dried and then resuspended in TE.

Digestion of the Engineered Plasmid

Since some restriction enzymes do not efficiently digest single strand plasmids, the strategy is to convert a portion of the engineered FR1-Constant region into dsDNA by hybridizing an oligonucleotide at that location and thereby allow digestion.

Single stranded engineered plasmid was mixed in sterile water with a 500 molar excess of a single stranded DNA primer complementary to the “FR1 variable-constant region” portion of the vector. The complementary primers used were the coding strand oligonucleotides used above to generate the vector inserts. This was then incubated in a 70° C. heatblock. The heatblock is then turned off and the temperature allowed to slowly cool to below 37° to allow primer annealing. Restriction endonuclease buffer and enzymes are then added. Plasmids are digested for 1 hour at the appropriate temperature (37° C. for Hinc II, 25° C. for Sma I). Single stranded DNA was then precipitated using ethanol. The cleaved plasmids are purified on a 1% agarose gel and then recovered from the gel slice using the Agarase purification method (Roche Molecular Biochemicals, Indianapolis, Ind.).

Reverse Transcription of cDNA Into the Cleaved Plasmid

Purified linear single stranded vector is combined with mRNA collected from a pool of human B cells in sterile DEPC treated water, heated to 70° C. and cooled to allow primer region annealing. First strand cDNA initiates from the constant region primer of the cleaved plasmid. Reverse transcription terminates at the annealed FR1 collar sequence of the cleaved plasmid. T4 DNA ligase seals the nick between the newly synthesized cDNA and the FR1 collar sequence of the vector to generate a plasmid vector containing the newly formed single stranded DNA.

Different “families” of light chain or heavy chain genes, as specified by the FR1 region, can be reverse transcribed in separate reactions with the appropriate engineered plasmids. After reverse transcription, products are pooled, keeping light and heavy chains separate, for transformation into bacteria. Following amplification in bacteria, light and heavy chain genes can be combined by a subsequent cloning step to allow for greater recombination and antibody diversity.

Transformation into E.coli

After cDNA synthesis, the single stranded plasmid vector containing the first strand cDNA is directly transformed into E.coli by electroporation. The bacteria is cultured to both convert the single stranded plasmids to double stranded DNA as well as amplify the copy number. Initial testing indicated that single stranded DNA could be directly transformed into bacteria, but at an approximate ⅓ of the efficiency of that observed with double stranded DNA.

Those skilled in the art will recognize, or be able to ascertain using no more than routine experimentation, many equivalents to the specific embodiments described specifically herein. Such equivalents are intended to be encompassed in the scope of the following claims.

We claim:

1. A plasmid comprising:
a primer sequence capable of annealing to a first portion of nucleic acid encoding a polypeptide;
a collar sequence capable of annealing to a second portion of the nucleic acid encoding a polypeptide, the second portion of the nucleic acid encoding a polypeptide being at least 20 nucleotides removed from the first portion of the nucleic acid encoding a polypeptide; and
at least one restriction site located between the primer and collar sequences.

2. A plasmid as in claim 1 wherein the primer and collar sequences are capable of annealing to first strand cDNA encoding a polypeptide.

3. A plasmid as in claim 1 wherein the primer and collar sequences are capable of annealing to mRNA encoding a polypeptide.

4. A plasmid as in claim 1 wherein the primer and collar sequences are capable of annealing to mRNA encoding at least a portion of an antibody.

5. A plasmid as in claim 1 wherein the collar sequence is capable of annealing to a portion of the nucleic acid encoding a polypeptide that is remote in the 5′ direction from the portion of the nucleic acid to which the primer sequence is capable of annealing.

6. A host cell transformed with a plasmid of claim 1.

7. A method of producing nucleic acid encoding a polypeptide, the method comprising the steps of:
a) providing a plasmid containing a primer sequence adapted to anneal to a first portion of nucleic acid encoding a polypeptide and a collar sequence adapted to anneal to a second portion of the nucleic acid encoding a polypeptide, the second portion of the nucleic acid encoding a polypeptide being at least 20 nucleotides removed from the first portion of the nucleic acid encoding a polypeptide;
b) cleaving the plasmid at one or more restriction sites, the one or more restriction sites being located on the plasmid between the primer and collar sequences; and
c) exposing the cleaved plasmid to nucleic acid encoding a polypeptide in an aqueous environment containing nucleotides and one or more enzymes selected from the group consisting of polymerase, reverse transcriptase, ligases and combinations thereof to form a nucleic acid strand that is complementary to the nucleic acid encoding a polypeptide, the complementary nucleic acid strand being formed between the primer and collar sequences whereby the plasmid is ligated and circularized.

8. The method of claim 7 further comprising the step of removing the complementary nucleic acid strand from the plasmid.

9. A method as in claim 7 wherein the step of cleaving the plasmid comprises exposing the plasmid to an oligonucleotide that hybridizes to the one or more restriction sites on the plasmid prior to exposing the plasmid to a restriction enzyme.

10. A method as in claim 7 wherein the step of exposing the cleaved plasmid to a nucleic acid encoding a polypeptide comprises exposing the cleaved plasmid to mRNA encoding a polypeptide.

11. A method as in claim 10 wherein the cleaved plasmid is exposed to mRNA encoding at least a portion of an antibody.

12. A method as in claim 7 wherein the step of exposing the cleaved plasmid to a nucleic acid encoding a polypeptide comprises exposing the cleaved plasmid to first strand cDNA.

13. A host cell transformed with a circularized plasmid produced in step c) of claim 7.

14. A polypeptide produced using the plasmid of claim 1.

15. A polypeptide produced using the circularized plasmid produced in step c) of claim 7.

16. A plasmid comprising:
a primer sequence adapted to anneal to a first portion of a nucleic acid encoding a polypeptide;
a collar sequence adapted to anneal to a second portion of the nucleic acid encoding a polypeptide, the second portion of the nucleic acid encoding a polypeptide being at least 20 nucleotides removed from the first portion of the nucleic acid encoding a polypeptide; and
a nucleic acid strand that is complementary to the nucleic acid encoding a polypeptide, the complementary nucleic acid strand being formed in situ between the primer and collar sequences.

17. A plasmid as in claim 16 wherein the primer and collar sequences are adapted to anneal to first strand cDNA encoding a polypeptide.

18. A plasmid as in claim 16 wherein the primer and collar sequences are adapted to anneal to mRNA encoding a polypeptide.

19. A plasmid as in claim 16 wherein the primer and collar sequences are adapted to anneal to a portion of mRNA encoding at least a portion of an antibody.

20. A host cell transformed with a plasmid of claim 16.

21. A polypeptide produced using a plasmid of claim 16.

22. A plasmid containing a nucleic acid sequence encoding at least a portion of a polypeptide prepared in accordance with the method of claim 7.

23. A plasmid comprising:
a downstream primer capable of annealing to a first portion of mRNA encoding at least a portion of an antibody;
an upstream collar sequence capable of annealing to a second portion of the mRNA encoding at least a portion of an antibody; and
at least one restriction site located between the downstream primer and upstream collar sequence.

24. A plasmid as in claim 23 wherein the upstream collar sequence is capable of annealing to a portion of the mRNA encoding a framework region of an antibody.

25. A plasmid as in claim 23 wherein the upstream collar sequence is capable of annealing to a leader sequence of the mRNA encoding an antibody.

26. A plasmid as in claim 23 wherein the upstream collar sequence is capable of annealing to a portion of the mRNA encoding a framework region associated with a light chain of an antibody.

27. A plasmid as in claim 23 wherein the upstream collar sequence is capable of annealing to a portion of the mRNA encoding a framework region associated with a heavy chain of an antibody.

28. A plasmid as in claim 23 wherein the downstream primer is capable of annealing to a portion of the mRNA encoding a constant region of an antibody.

29. A plasmid as in claim 23 wherein the downstream primer is capable of annealing to a portion of the mRNA encoding a constant region associated with a light chain of an antibody.

30. A plasmid as in claim 23 wherein the downstream primer is capable of annealing to a portion of the mRNA encoding a framework two (FR2), framework three (FR3) or framework four (FR4) region associated with a light chain of an antibody.

31. A plasmid as in claim 23 wherein the downstream primer is capable of annealing to a portion of the mRNA encoding a constant region associated with a heavy chain of an antibody.

32. A plasmid as in claim 23 wherein the downstream primer is capable of annealing to a portion of the mRNA encoding a framework two (FR2), framework three (FR3) or framework four (FR4) region associated with heavy chain of an antibody.

33. A plasmid as in claim 23 wherein the downstream primer comprises SEQ. ID. NO: 4.

34. A plasmid as in claim 23 wherein the downstream primer comprises SEQ. ID. NO: 8.

35. A plasmid as in claim 23 wherein the upstream collar sequence comprises SEQ. ID. NO: 3.

36. A plasmid as in claim 23 wherein the upstream collar sequence comprises SEQ. ID. NO: 7.

37. A host cell transformed with a plasmid of claim 23.

38. A method of producing nucleic acid encoding at least a portion of an antibody, the method comprising the steps of:
a) providing a plasmid containing a downstream primer adapted to anneal to a first portion of mRNA encoding at least a portion of an antibody and an upstream collar sequence adapted to anneal to a second portion of the mRNA encoding at least a portion of an antibody;
b) cleaving a plasmid at one or more restriction sites, the one or more restriction sites being located on the plasmid between the downstream primer and upstream collar sequence;
c) exposing the cleaved plasmid to the mRNA encoding at least a portion of an antibody in an aqueous environment containing reverse transcriptase and nucleotides to form a nucleic acid strand that is complementary to the mRNA, the complementary nucleic acid strand being formed between the downstream primer and upstream collar; and
d) ligating the complementary nucleic acid strand to the upstream collar whereby the plasmid is ligated and circularized.

39. The method of claim 38 further comprising the step of removing the complementary nucleic acid strand from the plasmid.

40. A method as in claim 38 wherein the step of cleaving the plasmid comprises exposing the plasmid to a restriction enzyme.

41. A method as in claim 40 wherein the step of cleaving the plasmid comprises exposing the plasmid to an oligonucleotide that hybridizes to the one or more restriction sites on the single strand plasmid prior to exposing the plasmid vector to a restriction enzyme.

42. A method as in claim 38 wherein the step of exposing the cleaved plasmid to the mRNA forms a complementary nucleic acid strand encoding at least a light chain of an antibody.

43. A method as in claim 38 wherein the step of exposing the cleaved plasmid to the mRNA forms a complementary nucleic acid strand encoding at least a heavychain of an antibody.

44. A method as in claim 39 wherein the step of removing the complementary nucleic acid strand from the plasmid comprises digesting the plasmid containing the complementary nucleic acid strand.

45. A method as in claim 44 wherein the step of digesting the plasmid containing the complementary nucleic acid strand comprises the steps of hybridizing a oligonucleotide to a portion of the single strand antibody DNA to provide a double stranded restriction site and exposing the double stranded restriction site to a restriction enzyme.

46. A method as in claim 38 wherein the step of providing a plasmid comprises providing a plasmid wherein the upstream collar sequence is adapted to anneal to a portion of the mRNA encoding a framework region of an antibody.

47. A method as in claim 38 wherein the step of providing a plasmid comprises providing a plasmid wherein the upstream collar sequence is adapted to anneal to a portion of the mRNA encoding a framework region associated with a light chain of an antibody.

48. A method as in claim 38 wherein the step of providing a plasmid comprises providing a plasmid wherein the upstream collar sequence is adapted to anneal to a portion of the mRNA encoding a framework region associated with a heavy chain of an antibody.

49. A method as in claim 38 wherein the step of providing a plasmid comprises providing a plasmid wherein the downstream primer is adapted to anneal to a portion of the mRNA encoding a constant region of an antibody.

50. A method as in claim 38 wherein the step of providing a plasmid comprises providing a plasmid wherein the downstream primer is adapted to anneal to a portion of the mRNA encoding a constant region associated with a light chain of an antibody.

51. A method as in claim 38 wherein the step of providing a plasmid comprises providing a plasmid wherein the downstream primer is adapted to anneal to a portion of the mRNA encoding a constant region associated with a heavy chain of an antibody.

52. A method as in claim 38 wherein the step of providing a plasmid comprises providing a plasmid wherein the upstream collar sequence comprises SEQ. ID. NO: 3.

53. A method as in claim 38 wherein the step of providing a plasmid comprises providing a plasmid wherein the upstream collar sequence comprises SEQ. ID. NO: 7.

54. A method as in claim 38 wherein the step of providing a plasmid comprises providing a plasmid wherein the downstream primer comprises SEQ. ID. NO: 4.

55. A method as in claim 38 wherein the step of providing a plasmid comprises providing a plasmid wherein the downstream primer comprises SEQ. ID. NO: 8.

56. A host cell transformed with a circularized plasmid produced in step d) of claim 38.

57. An antibody or antibody fragment produced using the plasmid of claim 23.

58. An antibody or antibody fragment produced using the circularized plasmid produced in step d) of claim 38.

59. A plasmid comprising:
a downstream primer adapted to anneal to a first portion of mRNA encoding at least a portion of an antibody;
an upstream collar sequence adapted to anneal to a second portion of the mRNA encoding at least a portion of an antibody; and
a nucleic acid strand that is complementary to the mRNA, the complementary nucleic acid strand being formed in situ between the downstream primer and upstream collar sequence to close the plasmid.

60. A plasmid as in claim 59 wherein the upstream collar sequence is adapted to anneal to a portion of the mRNA encoding a framework region of an antibody.

61. A plasmid as in claim 59 wherein the upstream collar sequence is adapted to anneal to a portion of the mRNA encoding a framework region associated with a light chain of an antibody.

62. A plasmid as in claim 59 wherein the upstream collar sequence is adapted to anneal to a portion of the mRNA encoding a framework region associated with a heavy chain of an antibody.

63. A plasmid as in claim 59 wherein the downstream primer is adapted to anneal to a portion of the mRNA encoding a constant region of an antibody.

64. A plasmid as in claim 59 wherein the downstream primer is adapted to anneal to a portion of the mRNA encoding a constant region associated with a light chain of an antibody.

65. A plasmid as in claim 59 wherein the downstream primer is adapted to anneal to a portion of the mRNA encoding a constant region associated with a heavy chain of an antibody.

66. A plasmid as in claim 59 wherein the upstream collar sequence comprises SEQ. ID. NO: 3.

67. A plasmid as in claim 59 wherein the upstream collar sequence comprises SEQ. ID. NO: 7.

68. A plasmid as in claim 59 wherein the downstream primer comprises SEQ. ID. NO: 4.

69. A plasmid as in claim 59 wherein the downstream primer comprises SEQ. ID. NO: 8.

70. A host cell transformed with a plasmid of claim 59.

71. An antibody or antibody fragment produced using the plasmid of claim 59.

72. A plasmid containing a nucleic acid sequence encoding at least a portion of an antibody prepared in accordance with the method of claim 38.

73. A plasmid as in claim 1 wherein two restriction sites that are the same or different are located between the downstream primer and upstream collar sequences.

74. A plasmid as in claim 23 wherein two restriction sites that are the same or different are located between the downstream primer and upstream collar sequence.

75. A library containing a repertoire of antibodies or antibody fragments made using the plasmid of claim 23.

76. A library containing a repertoire of antibodies or antibody fragments made using the method of claim 38.

77. A library containing a repertoire of antibodies or antibody fragments made using the plasmid of claim 59.

78. A method of producing nucleic acid encoding a polypeptide, the method comprising the steps of:
a) providing a plasmid containing a primer sequence adapted to anneal to a first portion of nucleic acid encoding a polypeptide;
b) cleaving the plasmid at one or more restriction sites, the one or more restriction sites being located on the plasmid to provide a cleaved plasmid having the primer at one end thereof and a free end; and
c) exposing the cleaved plasmid to nucleic acid encoding a polypeptide in an aqueous environment containing nucleotides and one or more enzymes selected from the group consisting of polymerase, reverse transcriptase, ligases and combinations thereof to form a nucleic acid strand that is complementary to the nucleic acid encoding a polypeptide, the complementary nucleic acid strand being formed attached to the primer and having a free end.

79. A method as in claim 78 further comprising the step of joining the free end of the complementary nucleic acid strand to the free end of the plasmid whereby the plasmid is circularized.

80. A method as in claim 79 wherein the step of joining comprises providing a bridging oligonucleotide.

81. The method of claim 78 further comprising the step of removing the complementary nucleic acid strand from the cleaved plasmid.

82. A method as in claim 78 wherein the step of exposing the cleaved plasmid to a nucleic acid encoding a polypeptide comprises exposing the cleaved plasmid to first strand cDNA.

83. A method as in claim 82 wherein the cleaved plasmid is exposed to antibody cDNA encoding at least a portion of an antibody.

84. A method as in claim 82 wherein the first strand cDNA is cleaved at a restriction site prior to being exposed to the cleaved plasmid.
